(19) United States
(12) Patent Application Publication
(10) Pub. No.: US 2023/0035127 A1
(43) Pub. Date: Feb. 2, 2023

(54) CULTURE AND DIFFERENTIATION OF PLURIPOTENT STEM CELLS

(71) Applicant: University of Maryland, College Park, College Park, MD (US)

(72) Inventors: Xiaoming He, Bethesda, MD (US); Bin Jiang, College Park, MD (US)

(21) Appl. No.: 17/814,044

(22) Filed: Jul. 21, 2022

Related U.S. Application Data
(60) Provisional application No. 63/224,283, filed on Jul. 21, 2021.

(51) Int. Cl. G01N 33/50; C12N 5/077

(52) U.S. Cl. CPC G01N 33/5061; C12N 5/0657; C12N 2513/00; C12N 2506/45; C12N 2503/02; C12N 2500/32; C12N 2500/12; C12N 2500/44; C12N 2501/115; C12N 2501/15; C12N 2501/415; C12N 2501/727

(57) ABSTRACT

Methods and compositions for the production of cardiomyocytes and cardiac organoids from the differentiation of pluripotent stem cells in three-dimensional (3D) culture are provided. Rock inhibitor, which has been ubiquitously used as a medium supplement to prevent apoptosis during handling and culture of pluripotent stem cells, compromises the capacity of cardiac differentiation of pluripotent stem cells in 3D culture at the most commonly used concentrations.

### CROSS REFERENCE TO RELATED APPLICATIONS

This application claims priority to provisional application U.S. Ser. No. 63/224,283, filed Jul. 21, 2021, which hereby is incorporated herein by reference in its entirety.

### GOVERNMENT SUPPORT

This invention was made with government support under R01EB023632 awarded by the National Institutes for Health and CBET1831019 awarded by the National Science Foundation. The government has certain rights in the invention.

### TECHNICAL FIELD

The present disclosure relates to cell differentiation including preparing stem cells that maintain pluripotency in two and three-dimensional cultures.

### BACKGROUND

Heart diseases are the leading cause of death in the United States. A major reason for this is that the human heart has very limited capacity to regenerate cardiomyocytes once they are damaged by some malfunction (e.g., ischemia), leading to myocardial infarction. Stem cell therapy has been considered as a promising strategy for treating heart diseases. It is well accepted now that functional/beating human cardiomyocytes can be differentiated only from human pluripotent stem cells (PSCs) including human embryonic stem cells (ESCs) and induced pluripotent stem cells (iPSCs), although mesenchymal stem cells may be used for cardiac regeneration via their cytokine effect and differentiation into some cardiac stromal cells. Human PSC-derived cardiomyocytes can be used as not only a therapeutic agent for treating heart diseases, but also a valuable tool for elucidating the etiology and pathogenesis of heart diseases and developing engineered heart tissues to test the cardiotoxicity of pharmaceutical drugs. There is an urgent need to develop a stable system to upscale the generation of human PSC-derived cardiomyocytes for both basic and translational applications.

### SUMMARY

Applicants have discovered that Rock inhibitor (RI), which has been ubiquitously used as a medium supplement to prevent apoptosis during handling and culture of human PSCs, including both ESCs and iPSCs, compromises the quality of PSCs in three-dimensional (3D) culture and their capacity of directed cardiac differentiation in 3D. By reducing the RI concentration for handling and culturing PSCs, cardiac differentiation is improved with the outset beating time (OBT) synchronized within 24 hours (versus 7 or more days for all contemporary protocols) and the time required for cardiac differentiation is shortened by at least 7 days.

Methods of maintaining pluripotency of stem cells in 3D culture are provided. The methods comprise culturing pluripotent stem cells in suspension to form an aggregate in a medium comprising 5 μM or less of a Rock inhibitor. In certain embodiments, the medium comprises from about 0.01 μM to about 5 μM of the Rock inhibitor. In certain embodiments, the medium comprises about 1 μM of the Rock inhibitor. In certain embodiments, the methods further compromise inducing differentiation of the pluripotent stem cells.

Methods of producing cardiomyocytes or cardiac organoids are also provided. The methods comprise culturing pluripotent stem cells in suspension to form an aggregate in a medium comprising 5 μM or less of a Rock inhibitor; culturing the aggregate of pluripotent stem cells in suspension in a medium without Rock inhibitor; and inducing cardiac differentiation of the aggregate of pluripotent stem cells, thereby producing cardiomyocytes or cardiac organoids.

Cardiomyocytes and cardiac organoids produced by the methods are provided. Therapeutic agents comprising the cardiomyocytes or cardiac organoids are also provided.

Further provided are methods for screening an agent for improving or diminishing cardiac function as well as methods for treating a disorder of a cardiac tissue.

While multiple embodiments are disclosed, still other embodiments will become apparent to those skilled in the art from the following detailed description, which shows and describes illustrative embodiments. Accordingly, the figures and detailed description are to be regarded as illustrative in nature and not restrictive.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1A is a schematic illustration showing Rock inhibitor (RI) at 10 μM induces early gastrulation-like heterogeneous differentiation of human iPSCs and compromises their cardiac differentiation in 3D. Reducing RI to 1 μM retains human iPSCs in solid inner cell mass-like spheroids with high pluripotency for efficient and homogeneous cardiac differentiation. FIG. 1B is a schematic illustration of the timing for making the 3D eiPSC (episomal iPSCs) spheroids in 4 days and subsequently differentiating the eiPSCs into 3D cardiac spheroids (i.e., organoids). Either 1, 5, or 10 μM Rock inhibitor (RI) was supplemented into the eiPSC medium for culturing the eiPSCs detached from 2D surface into eiPSC spheroids under 3D suspension culture. The eiPSC spheroids on day 0 were cultured with CHIR99021 and BIO in a mesoderm induction medium for 1 day to induce mesoderm differentiation. Afterward, KY02111 and XAV939 were supplemented in the cardiac maintenance medium to culture the spheroids for 6 days for cardiac commitment to obtain the cardiac spheroids. Lastly, cardiac maintenance medium without KY02111 and XAV939 was used to culture the cardiac spheroids for cardiac maturation. The cardiac spheroids were observed to start to beat on days 6-6.5.

FIG. 2A-C shows the morphology of eiPSC spheroids on days −3 and 0 under 3D suspension culture with 1 and 10 μM RI. FIG. 2A shows the eiPSC spheroids in the 10 μM RI group have an evident archenteron-like cavity on day −3, although it becomes less evident on day 0, probably due to the inward growth of the cells in the shell. No evident core-shell structure is observable for the eiPSC spheroids in the 1 μM RI group on all days. FIG. 2B shows scanning electron microscopy (SEM) images of eiPSC spheroids on day −3. The archenteron-like cavity in the eiPSC aggregate of the 10 μM RI group is also evident. Scale bars: 50 μm. FIG. 2C shows the size (in diameter) distributions of the human eiPSC spheroids on day 0 before cardiac differentiation for both the 1 and 10 μM RI groups. A total of 416 (=184+120+112) and 555 (=196+171+188) spheroids from 3 independent runs were used for the 1 and 10 μM RI groups, respectively.

FIG. 3 shows characterization of teratomas grown from eiPSC spheroids in vivo. The eiPSCs grown in 3D suspension culture with different RI concentrations (1 and 10 μM) were collected on day 0 and injected subcutaneously into NOD/SCID mice. The formation of teratoma in vivo was confirmed by histology analysis with hematoxylin and eosin (H&E) staining, which shows the presence of tissues of all the three germ layers including neural epithelium (ectoderm) with hypernucelated neuroectodermal structures, the nidus of cartilage (mesoderm) with surrounding condensed mesenchymal cells, and gut epithelium (endoderm) with subnuclear vacuoles and tube structure. Scale bar: 100 μm.

FIG. 4A-E shows characterization of cardiac spheroids differentiated from eiPSCs made with 1, 5, and 10 μM RI in the medium before cardiac differentiation. FIG. 4A is quantitative data showing the percentage of beating cardiac spheroids over time during a period of 13.5 days post the initiation of cardiac differentiation on day 0. Approximately 97% of the cardiac spheroids in the 1 μM RI group start to beat within 24 h, while 44.3% of cardiac spheroids were observed to beat over more than 7 days (from day 6-6.5 to day 13.5) for the 10 μM RI group, and 61% of cardiac spheroids were observed to beat over more than 3 days (from day 6-6.5 to day 9.5) for the 5 μM RI group. **, p<0.01. FIG. 4B shows the morphology of cardiac spheroids (obtained on day 13.5 of cardiac differentiation) that were transferred into a regular cell culture petri dish and cultured for 3 days. The cardiac spheroids in the 1 μM RI group maintain the spheroidal shape with few cells attached to the surface. Many cells in the cardiac spheroids in the 10 μM RI group migrate out and attach to the petri dish with fibroblast-like and neuronal-like morphology. Scale bars: 100 μm. FIG. 4C is quantitative data showing few cardiac spheroids in the 1 μM RI group attach on the surface while >60% cardiac spheroids in the 10 μM RI group attach while the 5 μM RI group show 41% cardiac spheroids attach on the surface. **, p<0.01. FIG. 4D is immunostaining data showing there were cells positive for the neural-specific markers (TUJ-1 and MUSASHI) in the cardiac spheroids of the 10 μM RI group, while it is not observable for the cardiac spheroids of the 1 μM RI group. Scale bars: 100 μm. FIG. 4E is immunostaining data showing much higher expression of the cardiac-specific protein markers (cTnT and CX-43) in the cardiac spheroids (collected on day 12.5) of the 1 μM RI group than the 10 μM RI group. Scale bars: 100 μm.

FIG. 5A-C shows cTnT protein expression of cardiac spheroids decreases with increasing RI concentration for culturing the human eiPSCs. FIG. 5A shows typical flow cytometry peaks demonstrating the percentage of cTnT-positive cells in cardiac spheroids on day 15 decreases as the RI treatment concentration increases from 1 to 5 and to 10 μM. FIG. 5B shows quantitative data from the flow cytometry analyses showing the percentage of cTnT-positive cells in all three groups of cardiac spheroids on day 15. FIG. 5C shows quantitative data from the flow cytometry analyses showing the cTnT protein expression (represented by the mean fluorescence intensity of cTnT staining) in all three groups of cardiac spheroids on day 15. The 1 μM RI group has significantly more cTnT-positive cells and higher cTnT protein expression than both the 5 and 10 μM RI groups. **, p<0.01.

FIG. 6 shows a heat-map of the global genomic transcription at different stages of cardiac differentiation. The 3D undifferentiated (U), cardiac commitment (C), and cardiac maturation (M) stages for both the 1 (one, O) and 10 (ten, T) μM RI groups together with the 2D cultured eiPSCs were analyzed. The data show changes in gene expression among different samples at different stages (2D, UO, UT, CO, CT, MO, and MT) of the procedure for 3D iPSC culture and cardiac differentiation.

FIG. 7 shows a Venn diagram of gene expression. The data show the number of genes that were uniquely (in the non-overlapping regions) expressed within each group (MO/MT/2D, CO/CT/2D, UO/UT/2D), and the number of genes (in the overlapping regions) that were co-expressed in two or three groups.

FIG. 8A-C shows volcano plots of the overall distribution of differentially expressed genes. FIG. 8A shows UT vs. 2D, UO vs. 2D, and UO vs. UT. FIG. 8B shows CT vs. 2D, CO vs. 2D, and CO vs. CT. FIG. 8C shows MT vs. 2D, MO vs. 2D, and MO vs. MT. The threshold of differential gene expression is: |log₂ (fold change)|>1 and padj value <0.05. The number of differential gene expressions including the up-regulated genes and down-regulated genes is listed below the plots.

FIG. 9A-F shows transcriptomic analysis of cardiac differentiation of eiPSCs cultured with 1 vs. 10 μM RI in the medium before cardiac differentiation. FIG. 9A-D shows Gene Ontology (GO) enrichment histogram displaying the top significantly upregulated differentially expressed gene groups. The adjusted p value (padj) is less than 0.05 for all the genes where the count indicates the number of enriched genes. log₂FC: log₂ (fold change). FIG. 9A shows GO enrichment histogram displaying the top 18 significantly upregulated differentially expressed gene groups for MO with respect to MT (MO vs. MT). The boxes indicate the top 2 groups of upregulated genes were enriched to heart development and muscle system process. FIG. 9B shows GO enrichment histogram displaying the top 18 significantly downregulated differentially expressed gene groups for MO vs. MT. The boxes indicate the top 2 groups of downregulated genes were enriched to forebrain development and axon development. FIG. 9C shows GO enrichment histogram displaying the top 18 significantly upregulated differentially expressed gene groups for MT vs. 2D. The boxes indicate the top 2 groups of upregulated genes were enriched to skeletal system development and heart development. Others were enriched to heterogeneous tissue development including the urogenital/renal system development, eye, cartilage, and angiogenesis, as indicated by asterisks. FIG. 9D shows GO enrichment histogram displaying the top 18 significantly upregulated differentially expressed gene groups for MO vs. 2D. The box indicates the top upregulated gene group is enriched to heart development. FIG. 9E is a heat-map displaying the transcriptional differences between the MT, MO, and 2D groups. The pluripotency genes were downregulated in both MT and MO; and heart development genes associated with sarcomere maturation and ion channels were upregulated in MO compared to MT, while more genes associated with heterogeneous tissue development including forebrain, axon, lung, ear, gut, kidney were upregulated in MT. The positive and negative numbers represent up- and downregulation, respectively. FIG. 9F is a heat-map displaying the transcriptional differences among the UO, 2D, UT, CO, CT, MO, and MT groups. The genes associated with the BMP signaling pathway were upregulated in UO compared to UT, while the RNA transcription and metabolism genes were downregulated in UO and upregulated in UT (indicated in the dashed yellow box). Subsequently, genes enriched to heart development of cardiac commitment show more early up-regulation in CO than CT (indicated in the dashed green box), which carries further into the cardiac maturation stage (MO vs. MT, in the dashed black box). The positive and negative numbers represent up- and downregulation, respectively.

FIG. 10 is a heat-map showing the transcriptional changes between 2D, UO, UT, MO, and MT groups. The numbers were for log₂ (fold change) and the red and blue represent up- and downregulation, respectively.

FIG. 11A-D shows transcriptomic analysis of the eiPSC spheroids before cardiac differentiation. FIG. 11A is a GO enrichment histogram displaying the top 18 significantly upregulated differentially expressed gene groups for UT vs. 2D. The top group of upregulated gene expression is enriched to heart development, in addition to other groups enriched to gastrulation, endoderm development, and pattern-specific process, as indicated by the asterisks. FIG. 11B is a GO enrichment histogram displaying the top 18 significantly upregulated differentially expressed gene groups for UO vs. 2D. The top group of upregulated gene expression is enriched to the negative regulation of phosphorylation, in addition to other groups enriched to the negative regulation of cell migration, cell motility, and SMAD signal transduction. There were two groups of genes enriched to myoblast differentiation and BMP signaling pathway, as indicated by the asterisks. FIG. 11C is a GO enrichment histogram displaying the top 18 significantly upregulated differentially expressed gene groups for UO vs. UT. The top two groups of upregulated gene expression were enriched to cell adhesion molecule binding and heart development (indicated by the boxes), in addition to other groups enriched to the cell-substrate junction and cell-cell junction. FIG. 11D is a GO enrichment histogram displaying the top 18 significantly downregulated differentially expressed gene groups for UO vs. UT. The top group of downregulated gene expression is enriched to spliceosomal complex formation, in addition to other groups enriched to structural constituents of ribosomes, response to zinc and cadmium, RNA transcription, and metabolism. The adjusted p value (padj) is less than 0.05 for all the genes in this figure. The count indicates the number of enriched genes.

FIG. 12 is a heat-map displaying the transcriptional changes between the CO, CT, and 2D groups. The numbers were for log₂ (fold change) and the red and blue represent up- and downregulation, respectively. The data indicate that CT has less cardiac commitment gene expression for heart morphogenesis than CO, and its hierarchy is similar to the 2D control, further showing CT has less heart development than CO.

FIG. 13 is a schematic illustration of an embryo at the blastocyst and early gastrulation stages. The pluripotent embryonic stem cells are in the solid-like inner cell mass (indicated by the dashed blue line). After further differentiation into the early gastrulation stage with invagination, the cells differentiated from the pluripotent inner cell mass are in a shell/sheet-like structure, including the epiblast and hypoblast (indicated by the dashed red line). The schemes of the blastula and the early gastrulation were regenerated according to the CC-BY license employed by Smart Servier Medical Art.

FIG. 14A-E shows characterization of the eiPSCs under 3D suspension culture with 1 vs. 10 μM RI. FIG. 14A shows representative peaks from flow cytometry analyses showing higher expression of the pluripotency protein markers OCT4, NANOG, and SSEA-4 and lower expression of the ectoderm protein marker NESTIN in the eiPSCs (collected on day 0) from the 1 μM RI group than 10 μM RI group. FIG. 14B shows quantitative data from the flow cytometry analyses showing cells in the 1 μM RI group have a significantly higher expression (represented by the mean fluorescence intensity) of all the three pluripotency markers and lower expression of the ectoderm maker than cells in the 10 μM RI group. Furthermore, significantly more cells were positive for two of the three pluripotency markers (OCT4 and NANOG) and significantly fewer cells were positive for the ectoderm marker in the 1 μM RI group than the 10 μM RI group. FIG. 14C shows immunostaining data showing high expression of all the three pluripotency markers (OCT-4, NANOG, and SSEA-4) and no evident expression of the ectoderm marker NESTIN in eiPSCs (collected on day 0) of the 1 μM RI group. FIG. 14D shows immunostaining data showing evident expression of not only the three pluripotency markers but also the ectoderm marker in the eiPSCs (collected on day 0) of the 10 μM RI group. FIG. 14E shows immunostaining data showing cells positive for the WNT4 and CD44 (which are markers for urogenital system and cartilage development, respectively) in the cardiac spheroids of the 10 μM RI group, but not the 1 μM RI group. All the cardiac spheroids were collected on Day 12.5 post-cardiac differentiation. Scale bars: 100 μm. *, p<0.05, and **, p<0.01.

FIG. 15A-C shows a high concentration of RI induces ectodermic differentiation of eiPSCs in 3D. FIG. 15A shows morphology of 2D monolayer of eiPSCs derived by culturing for 2 days in 2D the eiPSCs spheroids (on day 0) obtained with 0, 1, and 10 μM RI for 3D suspension culture. Low- and high-magnification images are given in the top and bottom rows, respectively. There were more spiky cells in the peripheral of the eiPSC colonies in the 10 μM RI group and the cells inside the colonies in the 10 μM RI group were not as compact as those in the 0 or 1 μM RI group. The eiPSC colonies in the 1 μM RI group contain compacted cells with minimal spiky cells in the peripheral, similar to the eiPSC colonies in the 0 μM RI group. FIG. 15B shows immunostaining of OCT4 and NESTIN on the eiPSCs cultured in 2D monolayer from the 0, 1, and 10 μM RI groups. The eiPSC colonies in the 10 μM RI group show loose cell contact with spiky morphology on the outer boundary and positive for both the neural protein marker NESTIN and the pluripotency protein marker OCT-4. In contrast, the eiPSC colonies of the 1 μM RI group were typical in terms of morphology with compacted cells, negative for the neural protein marker NESTIN, and positive for pluripotent gene marker OCT-4, which were similar to the eiPSC colonies without RI treatment (i.e., the 0 μM RI group). FIG. 15C shows immunostaining of NANOG and TUJ-1 on the eiPSC colonies in the 0, 1, and 10 μM RI under 2D monolayer culture. The eiPSC colonies in the 10 μM RI group show loosely connected cells with spiky morphology, positive expression for neural protein marker TUJ-1, and negative expression for pluripotency protein marker NANOG. In contrast, the eiPSC colonies in the 1 μM RI group were typical in terms of morphology with compact cells, negative expression for the neural protein marker TUJ-1, and positive expression for the pluripotency protein marker NANOG, which were similar to the eiPSCs without RI treatment (i.e., the 0 μM RI group). Scale bars: 100 μm.

FIG. 16A-D shows characterization of the stage-specific protein markers for cardiac differentiation of eiPSC spheroids from the 1 μM RI culture. FIG. 16A is immunostaining data showing the cells in the spheroids on day 1.5 were positive for the mesoderm specific gene marker BRACHYURY, indicating successful mesoderm induction. Scale bar: 100 μm. FIG. 16B is immunostaining data showing the cells in the spheroids on day 2.5 were positive for the early cardiac commitment specific protein marker NKX2.5, indicating successful induction of cardiac commitment to turn the eiPSC spheroids into cardiac spheroids. Scale bar: 100 μm. FIG. 16C is immunostaining data showing cells in the spheroids on day 12.5 were positive for the cardiac-specific protein markers α-ACTININ for sarcomeres and DESMIN for intermediate filaments that integrate sarcolemma and Z disks. The cardiac spheroids were filled with sarcomeres and intermediate filaments indicating high development of myofibrils, as shown in the zoom-in view of the merged image. Scale bars: 50 μm. FIG. 16D is flow cytometry analyses showing highly positive expression for the stage-specific protein markers including BRACHYURY (˜100%) on day 1.5; NKX2.5 (˜100%) on day 2.5; and cTnI (93.9%), α-ACTININ (95%) on day 12.5; while the expression of neural-specific protein markers MUSASHI-1 and TUJ-1 on day 12.5 is negative.

FIG. 17 shows flow cytometry analyses of cells in the cardiac spheroids of the 1 μM RI group with neural markers. The data show negligible expression of neural-specific protein markers MUSASHI-1 and TUJ-1 in the cells of cardiac spheroids (collected on day 12.5) of the 1 μM RI group.

FIG. 18A-G shows ultrastructural and functional analysis of the cardiac spheroids differentiated from eiPSC spheroids from the 1 μM RI culture. All cardiac spheroids were collected on day 12.5. FIG. 18A is a transmission electron microscopy (TEM) image showing a well-extended myofibril (MF) with a sarcoplasmic reticulum (SR), and multiple mitochondria located nearby in the cardiac spheroids. Scale bar: 2 μm. FIG. 18B is a zoom-in view of the dashed box area in FIG. 18A showing well-organized sarcomere (Sm) with aligned Z lines (ZL) in the myofibril. Scale bar: 500 nm. FIG. 18C is a TEM image showing abundant mitochondria, sarcoplasmic reticula, intercalated disc (iCD), and gap junctions in the cardiac spheroids. Scale bar: 250 nm. FIG. 18D is a TEM image showing abundant mitochondria, sarcoplasmic reticula, and two nuclei (Nu) in a cardiomyocyte in the cardiac spheroids, indicating the formation of multinucleated cardiomyocytes. Scale bar: 500 nm. FIG. 18E shows representative calcium transients of cardiac spheroids on day 12.5 before (control) and after treated with cardiac drugs isoproterenol (ISO, 1 μM) that increase the rate of heartbeat and propranolol (PRO, 1 μM) that decreases the rate of heartbeat, showing the responsiveness of the cardiac spheroids to the cardiac drugs. F is the fluorescence intensity of calcium stain, F₀ is the fluorescence intensity of calcium stain at the resting state of the cardiac spheroids, and ΔF=(F−F₀) is the change of the fluorescence intensity of calcium stain from the resting state. FIG. 18F shows quantitative data on the beating frequency of cardiac spheroids collected on day 12.5 before (control) and after treated the cardiac drugs ISO (1 μM) and PRO (1 μM), showing ISO and PRO increase and decrease the beating frequency of the cardiac spheroids, respectively. *, p<0.05, and **, p<0.01. FIG. 18G shows morphology of the construct of the 7% GelMA hydrogel suspended with cardiac spheroids (collected on day 5 post-initiation of cardiac differentiation) from the 1 μM RI group without culture and after cultured for 2 (on day 7) and 5 days (on day 10). The cardiac spheroids fuse together to beat synchronously after the 2-5 days of culture in the GelMA hydrogel. Scale bar: 200 μm.

FIG. 19A-E shows validation of the optimized protocol for cardiac differentiation with the IMR90-1 human iPSCs. FIG. 19A is quantitative data showing the percentage of beating IMR90-1 cardiac spheroids over time during a period of 13.5 days post initiation of cardiac differentiation. Approximately 98% of the IMR90-1 cardiac spheroids in the 1 μM RI groups start to beat within 24 h, while 34.1% of the IMR90-1 cardiac spheroids were observed to beat over 7 days (from day 6-6.5 to day 13.5) for the 10 μM RI group. FIG. 19B is quantitative data from flow cytometry analyses showing a significantly higher percentage of cTnT (cardiac-specific marker) positive cells and a significantly lower percentage of MUSASHI (neural-specific marker) cells in the cardiac spheroids (collected on day 12.5) from the 1 μM RI group than the 10 μM RI group. Moreover, the expression of MUSASHI in the cardiac spheroids of the 1 μM RI group is negligible while it is evident in the cardiac spheroids of the 1 μM RI group. FIG. 19C is representative peaks of flow cytometry analyses showing a comparison of the expression of cTnT and MUSASHI in the cardiac spheroids (collected on day 12.5) from the 1 and the 10 μM RI groups. FIG. 19D is immunostaining data showing cells positive for the neural gene markers (TUJ-1 and MUSASHI) were evident in the IMR90-1 cardiac spheroids (collected on day 12.5) from the 10 μM RI group, while it is not observable for the 1 μM RI group. FIG. 19E is immunostaining data showing reduced expression of cTnT and CX-43 in the IMR90-1 cardiac spheroids (collected on day 12.5) from the 10 μM RI group, while their expression is higher in the IMR90-1 cardiac spheroids (collected on day 12.5) from the 1 μM RI group. Scale bars: 100 μm. **, p<0.01.

FIG. 20A-B shows the effect of RI on the survival/yield of eiPSCs in 3D suspension culture. FIG. 20A shows increased concentration of RI from 0-10 μM enhances the yield of eiPSCs under 3D suspension culture. Using the same number of 2D cultured eiPSCs in clumps for the 3D suspension culture on day −4, the number of eiPSCs (spun down at the bottom of the centrifuge tubes) obtained after 4 days of 3D culture on day 0 is the highest for the 10 μM RI group, followed by the 1 and then the 0 μM RI groups. FIG. 20B is quantitative data showing that, although the number of eiPSCs obtained on day 0 in the 1 μM RI group is significantly less than that in the 10 μM RI groups, it is significantly more than that for the 0 μM RI group. **, p<0.01.

### DETAILED DESCRIPTION

The present disclosure relates to the surprising discovery that Rock inhibitor (RI), used ubiquitously to improve the survival and yield of PSCs, induces early gastrulation-like change to PSCs in 3D culture and causes their heterogeneous differentiation into all the three germ layers (i.e., ectoderm, mesoderm, and endoderm) at the commonly used concentration (10 μM). This greatly compromises the quality of PSCs for homogeneous 3D cardiac differentiation. By reducing the RI to 1 μM for 3D culture, the PSCs retain high pluripotency and high quality in inner cell mass-like solid 3D spheroids. Consequently, the beating efficiency of 3D cardiac differentiation can be improved to more than 95% in about 7 days (compared to less than about 50% in 14 days for the 10 μM RI condition). The outset beating time (OBT) of all resultant cardiac organoids is synchronized within only 1 day and they form a synchronously beating 3D construct after 5-day culture, showing high homogeneity (in terms of the OBT) in functional maturity of the cardiac organoids. The resultant cardiomyocytes are of high quality with key functional ultrastructures and highly responsive to cardiac drugs.

Unless defined otherwise, all technical and scientific terms used herein have the same meaning as commonly understood by one skilled in the art to which embodiments of the disclosure pertain. Many methods and materials similar, modified, or equivalent to those described herein can be used in the practice of the embodiments of the present disclosure without undue experimentation, the preferred materials and methods are described herein.

It is to be understood that all terminology used herein is for the purpose of describing particular embodiments only, and is not intended to be limiting in any manner or scope. For example, as used in this specification and the appended claims, the singular forms “a”, “an”, and “the” can include plural referents unless the content clearly indicates otherwise. Similarly, the word “or” is intended to include “and” unless the context clearly indicates otherwise. The word “or” means any one member of a particular list and also includes any combination of members of that list. Further, all units, prefixes, and symbols may be denoted in their SI accepted form.

Numeric ranges recited within the specification are inclusive of the numbers defining the range and include each integer within the defined range. Throughout this disclosure, various aspects are presented in a range format. It should be understood that the description in range format is merely for convenience and brevity and should not be construed as an inflexible limitation on the scope of the present disclosure or the associated claims. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The term “about”, as used herein, refers to variation in the numerical quantity that can occur, for example, through typical measuring and liquid handling procedures in the real world; through inadvertent error in these procedures; through differences in the manufacture, measurement quantifications (e.g., weight, volume, temperature, and time), source, or purity of the ingredients used to make the compositions or carry out the methods; and the like. Whether or not modified by the term “about”, the claims include equivalents to the quantities.

### Pluripotent Stem Cells

The term “stem cell” is used herein to refer to a mammalian cell that has the ability both to self-renew and to generate a differentiated cell type (see Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells (described below) can differentiate into germ layer-restricted stem cells (e.g., endodermal, mesodermal, ectodermal stem cells), which in turn can differentiate into cells that are further restricted (e.g., cardiac progenitors), which can differentiate into end-stage cells (i.e., terminally differentiated cells, e.g., cardiomyocytes), which play a characteristic role or function in a certain tissue type, and may or may not retain the capacity to proliferate further. Stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progenies.

The stem cells of interest are mammalian, where the term refers to cells isolated from any animal classified as a mammal, including humans, domestic and farm animals, and zoo, laboratory, sports, or pet animals, such as dogs, horses, cats, cows, mice, rats, rabbits, etc. In some embodiments, the mammal is a human and the mammalian cells are therefore human cells.

A “progenitor cell” is a type of stem cell that typically does not have extensive self-renewal capacity (i.e., the number of self-renewing divisions is limited), and often can only generate a limited number of differentiated cell types (e.g., a specific subset of cells found in the tissue from which they derive). Thus, a progenitor cell is differentiated relative to its mother stem cell, but can also give rise to cells that are further differentiated (e.g., terminally differentiated cells). For the purposes of the present disclosure, progenitor cells are those cells that are committed to a lineage of interest (e.g., a cardiac progenitor), but have not yet differentiated into a mature cell (e.g., a cardiomyocyte).

When a stem cell divides symmetrically, both resulting daughter cells are equivalent. For example, a stem cell may undergo a self-renewing symmetric division in which both resulting daughter cells are stem cells with an equal amount of differentiation potential as the mother cell. However, a symmetric division is not necessarily a self-renewing division because both resulting daughter cells may instead be differentiated relative to the mother cell. When a stem cell divides asymmetrically, the resulting daughter cells are different from one another. For example, if a stem cell undergoes a self-renewing asymmetric division, then one of the resulting daughter cells is a stem cell with the same amount of differentiation potential as the mother cell while the other daughter cell is differentiated relative to the mother cell (e.g., a more lineage restricted progenitor cell, a terminally differentiated cell, etc.). A stem cell may directly differentiate, or may instead produce a differentiated cell type through an asymmetric or symmetric cell division.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a mammal). Pluripotent cells are capable of forming teratomas and of contributing to ectoderm, mesoderm, or endoderm tissues in a living organism.

PSCs can be derived in a number of different ways. For example, embryonic stem cells (ESCs) are derived from the inner cell mass of an embryo (Thomson et al, Science. 1998 Nov. 6; 282(5391):1145-7) whereas induced pluripotent stem cells (iPSCs) are derived from somatic cells (Takahashi et. al, Cell. 2007 Nov. 30; 131(5):861-72; Takahashi et. al, Nat Protoc. 2007; 2(12):3081-9; Yu et. al, Science. 2007 Dec. 21:318(5858):1917-20. Epub 2007 Nov. 20). Because the term PSC refers to pluripotent stem cells regardless of their derivation, the term PSC encompasses the terms ESC and iPSC, as well as the term embryonic germ stem cells (EGSC), which are another example of a PSC. A human PSC can be referred to as an “hPSC”, an “hESC”, an “hEGSC”, and/or an “hiPSC”, depending on the context and the derivation of the PSC. PSCs may be in the form of an established cell line, they may be obtained directly from primary embryonic tissue, or they may be derived from a somatic cell. The methods described herein can be used to produce cardiomyocytes from any mammalian PSC population, including but not limited to an ESC population, an iPSC population, and/or an EGSC population.

By “embryonic stem cell” (ESC) is meant a PSC that was isolated from an embryo, typically from the inner cell mass of the blastocyst. ESC lines are listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). Stem cells of interest also include embryonic stem cells from other primates, such as Rhesus stem cells and marmoset stem cells. The stem cells may be obtained from any mammalian species, e.g. human, equine, bovine, porcine, canine, feline, rodent (e.g. mice, rats, hamster), primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In culture, ESCs typically grow as flat colonies with large nucleo-cytoplasmic ratios, defined borders and prominent nucleoli. In addition, ESCs express SSEA-3, SSEA-4, TRA-1-60, TRA-1-81, and Alkaline Phosphatase, but not SSEA-1. Examples of methods of generating and characterizing ESCs may be found in, for example, U.S. Pat. Nos. 7,029,913, 5,843,780, and 6,200,806, the disclosures of which are incorporated herein by reference. Methods for proliferating hESCs in the undifferentiated form are described in WO 99/20741, WO 01/51616, and WO 03/020920.

By “embryonic germ stem cell” (EGSC) or “embryonic germ cell” or “EG cell” is meant a PSC that is derived from germ cells and/or germ cell progenitors, e.g. primordial germ cells, i.e. those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al. (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235. the disclosures of which are incorporated herein by reference.

By “induced pluripotent stem cell” or “iPSC” it is meant a PSC that is derived from a cell that is not a PSC (i.e., from a cell that is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei on a 2D substrate. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPSCs may be found in, for example, U.S. Patent Publication Nos. US20090047263, US20090068742, US20090191159, US20090227032, US20090246875, and US20090304646, the disclosures of which are incorporated herein by reference. Generally, to generate iPSCs, somatic cells are provided with reprogramming factors (e.g., Oct4, SOX2. KLF4, MYC, Nanog, Lin28, etc.) known in the art to reprogram the somatic cells to become pluripotent stem cells.

The term “episomal induced pluripotent stem cells” or “eiPSCs” refers to somatic cells that are reprogrammed into induced pluripotent stem cells (iPSCs) using non-integrative episomal vector methods. eiPSCs are virus-free, which further enhances their translational value.

Compared to human ESCs, human iPSCs have no ethical concerns and are more easily accepted by society because they are not from human embryos. Of note, human iPSC banks may be established for generating human leukocyte antigen (HLA)-matched cell transplantation for known HLA types of donor and recipient, and the human iPSCs have great potential for allogeneic cell therapy besides autologous cell therapy.

By “somatic cell” it is meant that a cell of an organism that is not a germ cell. Thus, in the absence of experimental manipulation, a mammalian somatic cell does not ordinarily give rise to all types of cells in the body, although adult somatic stem cells do exist (e.g., lineage restricted progenitor cells)

### Rock Inhibitor and 3D Culture

Rho-kinase (Rock) inhibitor (RI) has been ubiquitously used to improve the survival and yield of iPSCs and ESCs at a concentration of 10 μM, which is the optimal concentration for preventing apoptosis of the PSCs under both 2D and 3D cultures. With all other conditions being kept consistent, using 10 μM RI in the medium for culturing the PSCs in 3D significantly improves the cell yield (i.e., number of viable cells) by about 3 and 2 times, compared to 0 or 1 μM RI, respectively. The use of 1 μM RI significantly increases the cell survival and yield compared to a 0 μM RI control. Unfortunately, RI at 10 μM induces uncontrolled spontaneous differentiation of PSCs into heterogeneous lineages including the ectoderm (e.g., neural and eye development), endoderm (e.g., lung and gut development), and non-cardiac mesoderm (e.g., urogenital and cartilage development) both before (i.e., on days −4 to 0) and after (i.e., after day 0) cardiac differentiation. The heterogeneous differentiation of PSCs cultured with 10 μM RI before cardiac differentiation is also evidenced by an archenteron-like cavity of the PSC spheroids, similar to the epiblast and hypoblast cells in the early gastrula. This uncontrolled spontaneous differentiation into heterogeneous lineages greatly compromises the efficiency and functional homogeneity (in terms of OBT) of directed or guided cardiac differentiation. In contrast, the use of 1 μM RI results in more than 95% beating cardiac organoids for cardiac differentiation of PSCs with the OBT being synchronized within 24 hours (compared to more than 7 days for the 10 μM RI group), indicating a highly efficient and homogeneous cardiac differentiation. This is attributed to the high pluripotency and high quality of the PSCs cultured with 1 μM RI in the solid inner cell mass-like spheroids.

Thus, in certain embodiments, the methods of the disclosure comprise the step of: (a) culturing pluripotent stem cells (e.g., single cells or clusters) in suspension to form an aggregate in a medium comprising 5 μM or less of a Rock inhibitor. In certain embodiments, the methods further comprise the step of: (b) culturing the aggregate of pluripotent stem cells in suspension in a medium without Rock inhibitor. Various media formulations are available and known in the art, and can be used to culture the PSCs. In certain embodiments, the medium is mTeSR1 or StemFlex.

As used herein, an “aggregate” refers to a three-dimensional association of cells in which the association is caused by cell-cell interaction rather than adherence to a substrate.

In one embodiment, the medium is supplemented with from about 0.01 μM to about 5 μM of the Rock inhibitor, from about 0.1 μM to about 4 μM of the Rock inhibitor, from about 0.5 μM to about 2 μM of the Rock inhibitor, or from about 0.75 μM to about 1.25 μM of the Rock inhibitor. In one embodiment, the medium is supplemented with about 0.5 μM of the Rock inhibitor, about 0.75 μM of the Rock inhibitor, about 1 μM of the Rock inhibitor, about 1.25 μM of the Rock inhibitor, about 1.5 μM of the Rock inhibitor, about 1.75 μM of the Rock inhibitor, or about 2 μM of the Rock inhibitor.

Examples of Rock inhibitors include Y27632 ((+)-(R)-trans-4-(1-aminoethyl)-N-(4-pyridyl) cyclo-hexanecarboxamide dihydrochloride), Fasudil (1-(5-Isoquinolinesulfonyl)homopiperazine), and Thiazovivin (N-Benzyl-2-(pyrimidin-4-ylamino)thiazole-4-carboxamide). In certain embodiments, the Rock inhibitor is Y27632.

The medium can be supplemented with a viscosity enhancer. The term “viscosity enhancer” refers to any substance that can increase the viscosity of a liquid such as a medium. The presence of a viscosity enhancer reduces the fusion of the PSC aggregates during the suspension culture. Viscosity enhancers include, for example, methylcellulose, carboxymethyl cellulose, hydroxypropyl cellulose, hydroxypropylmethyl cellulose, hydroxypropylmethyl cellulose acetate stearate, hydroxypropylmethyl cellulose phthalate, polyacrylic acid, polyvinyl alcohol, polyethylene glycol, alginate, xanthan gum, acacia, chitosan, and combinations thereof. In certain embodiments, the viscosity enhancer is methylcellulose.

In a specific embodiment, the 3D culture is prepared by the following protocol: PSC colonies under 2D culture at about 80% confluence are treated with Versene for 2 minutes, rinsed with isotonic (by default) phosphate-buffered saline (PBS), and further detached from the substrate by gentle pipetting. The detached PSCs are re-suspended in mTeSR1 with 5 μM or less Rock inhibitor. Afterward, the medium is supplemented with 0.35% (v/v) methylcellulose. The cell suspension is pushed through a cell strainer with 100 μm mesh size. Later, the suspension of PSC clumps or clusters is transferred into a petri dish for culture in a humidified incubator at 37° C. and 5% CO₂ for 2 days. Then, the medium is changed to mTeSR1 (supplemented with 0.35% methylcellulose) with no Rock inhibitor, to further culture for 2 days before differentiation.

In some embodiments, prior to 3D culture, the PSCs are cultured in a maintenance media on a substrate (i.e., 2D culture). In a specific embodiment, PSCs are cultured in a maintenance media by the following protocol: the PSCs are cultured in Matrigel-coated plates in a maintenance medium made of DMEM/F12 supplemented with bFGF (120 ng/ml), TGF-β (1 ng/ml), γ-aminobutyric acid (100 μg/ml), LiCl 30 (μg/ml), L-glutamine (100 μg/ml, Gibco), MEM non-essential amino acid (NEAA) solution (0.5%), NaHCO₃ (500 μg/ml), chemically defined lipid concentrate (1%), sodium selenite (50 ng/ml), bovine serum albumin (20 mg/ml), and β-mercaptoethanol (4 μl per 500 ml medium). The cells are passaged twice a week at a ratio between 1:4 and 1:5 with Versene consisting of 0.48 nM ethylenediaminetetraacetic acid (EDTA) in 1× (by default) phosphate buffered saline (PBS).

### Cardiac Differentiation

As used herein, a “cardiomyocyte” or “myocardial cell” is a terminally differentiated heart muscle cell. A “cardiomyocyte progenitor” is defined as a cell that is capable (without dedifferentiation or reprogramming) of giving rise to progeny that include cardiomyocytes. Such progenitors may express various cytoplasmic and nuclear markers typical of the lineage, including, without limitation, cardiac troponin I (cTnI), cardiac troponin T (cTnT), sarcomeric myosin heavy chain (MHC), GATA-4, Nkx2.5, N-cadherin, β1-adrenoceptor (β1-AR), ANF, the MEF-2 family of transcription factors, creatine kinase MB (CK-MB), myoglobin, or atrial natriuretic factor (ANF).

The pluripotent stem cells of the disclosure can be differentiated into cardiomyocytes or cardiac organoids. The terms “cardiac organoid” and “cardiac spheroid” are used interchangeably herein to refer to a 3D multicellular in vitro cardiac tissue that recapitulates aspects of the in vivo organ.

In certain embodiments, the method for inducing cardiac differentiation of a pluripotent stem cell comprises the steps of (1) culturing a pluripotent stem cell in a medium comprising a Wnt signaling activator and (2) culturing a pluripotent stem cell produced in step (1) in a medium comprising a Wnt signaling inhibitor.

The term “Wnt signaling activator” or “Wnt agonist” as used herein refers to a substance which activates the Wnt signaling pathway. Examples of the Wnt signaling activator include a glycogen synthase kinase 3β (Gsk-3β) inhibitor such as 6-bromoindirubin-3′-oxime (BIO) or CHIR99021. More than one Wnt signaling activator, for example, 2, 3, or 4 Wnt signaling activators may be used in combination.

The term “Wnt signaling inhibitor” or “Wnt antagonist” as used herein refers to a substance which inhibits the Wnt signaling pathway. Examples of the Wnt signaling inhibitor include compounds such as KY02111, IWP2, XAV939, and IWR1, and proteins such as IGFBP4 and Dkk1. More than one Wnt signaling inhibitor, for example, 2, 3, or 4 Wnt signaling inhibitors may be used in combination.

The medium used in the step (1), the step of culturing a pluripotent stem cell in a medium comprising a Wnt signaling activator, and the medium used in the step (2), the step of culturing a pluripotent stem cell produced in step (1) in a medium comprising a Wnt signaling inhibitor, may be any conventional medium used for cardiac differentiation of a pluripotent stem cell and the composition of the differentiation medium is not specifically limited. Examples of the medium include DMEM/F12-based medium, RPMI1640-based medium, or α-MEM-based medium for cardiac differentiation.

In the methods of the disclosure, the period from the start of culture in a medium for cardiac differentiation (i.e., culture for cardiac differentiation) to the start of step (1) or (2) and the periods of steps (1) and (2) of may be appropriately determined. Step (2) may be started just after the end of step (1), or after a certain period from the end of step (1). The Wnt signaling activator and the Wnt signaling inhibitor may be added at early and middle phases of cardiac differentiation of a pluripotent stem cell, respectively. The early phase of cardiac differentiation of a pluripotent stem cell means a stage at which differentiation of a pluripotent stem cell into mesoderm is induced and the expression of a mesoderm marker gene is increased. The middle phase of cardiac differentiation of a pluripotent stem cell means a stage at which differentiation of mesoderm into cardiac muscle is induced. Examples of the mesoderm marker includes T, MIXL1, and NODAL. For example, step (1) may be conducted at day 0 to day 2 or day 0 to day 3 of culture for cardiac differentiation, in other words, for 2 or 3 days from the start of culture for cardiac differentiation, and step (2) may be conducted, up until day 28 of culture for cardiac differentiation, for 2 days or more (specifically, for 2, 3, 4, 5, 6, 7, 8, 9, 10, 11, or 12 days), preferably for 3 to 10 days, more preferably for 4 to 10 days, still more preferably for 4 to 8 days, even more preferably for 4 to 6 days. Preferably, step (2) is conducted for 4 to 6 days up until day 10 of culture for cardiac differentiation, for example day 3 to day 9, day 3 to day 8, day 3 to day 7, day 4 to day 10, day 4 to day 9, or day 4 to day 8 of culture for cardiac differentiation.

Concentrations of the Wnt signaling activator and the Wnt signaling inhibitor are not particularly limited. When the Wnt signaling activator is BIO or CHIR99021, the Wnt signaling activator may be used at a final concentration of 100 nM to 100 μM, preferably 1 μM to 10 μM. When the Wnt signaling inhibitor is KY02111 or XAV939, the Wnt signaling inhibitor may be used, for example, at a final concentration of 0.5 to 20 μM, preferably 1 to 10 μM.

In a specific embodiment, PSCs are differentiated into cardiomyocytes by the following protocol: on day 0 of differentiation, the PSCs are induced into mesoderm by up-regulation of the Wnt signaling pathway using 8 μM CHIR99021 and 2 μM BIO in mesoderm induction medium for 1 day. The mesoderm induction medium is a mixture of DMEM/F12 and α-MEM (v/v, 1:1), containing 2% Knockout Serum Replacement (KOSR), 1 mM L-glutamine, 1% MEM non-essential amino acids (NEAA), and 0.1 mM β-mercaptoethanol. After 1 day, the cells are induced for cardiac commitment by down-regulating the Wnt signaling pathway using 10 μM KY02111 and 10 μM XAV939 in cardiac maintenance medium for 6 days with the medium being changed every other day. The cardiac maintenance medium is a mixture of RPMI1640 and α-MEM (v/v, 1:1), containing 5% fetal bovine serum (FBS). Starting from day 8, the cardiac maintenance medium without KY02111 and XAV939 is used, and it is changed every other day for cardiac maturation. Xenogeneic KOSR and FBS used in the media for cardiac differentiation and maintenance, respectively, may be replaced with materials of human origin to further improve translational value.

Differentiation into a cardiomyocyte may be detected from, for example, the number of beating cardiac organoids, expression of a cardiac marker, expression of an ion channel, a response to an electrophysiological stimulus, or the like. Examples of a cardiac marker include α-MHC, β-MHC, cTnT, α-actinin, and NKX2.5. Examples of the ion channel include HCN4, Nav1.5, Cav1.2, Cav3.2 HERG1b, and KCNQ1.

Therapeutic agents comprising the cardiomyocytes or cardiac organoids of the disclosure are provided. A “therapeutic agent” as used herein refers to any, composition useful for therapeutic or diagnostic purposes. The term as used herein is understood to mean any composition that is administered to a subject for the diagnosis, cure, mitigation, treatment, or prevention of a condition.

In vitro cardiomyocytes produced by the methods of the disclosure provide a source of donor cardiomyocytes for cell replacement in damaged hearts. Many forms of heart disease, including congenital defects and acquired injuries, are irreversible because they are associated with the loss of non-regenerative, terminally differentiated cardiomyocytes. Current therapeutic regimes are palliative, and in the case of end-stage heart failure, transplantation remains the last resort. However, transplantation is limited by a severe shortage of both donor cells and organs. In cases of myocardial infarction, 1 billion cells would potentially need to be replaced, highlighting the need for high-throughput and reproducible methodologies for de novo cardiomyocyte production.

As such, the cardiomyocytes may be used for tissue reconstitution or regeneration in a human patient or other subject in need of such treatment. The cells are administered in a manner that permits them to graft or migrate to the intended tissue site and reconstitute or regenerate the functionally deficient area. Special devices are available that are adapted for administering cells capable of reconstituting cardiac function directly to the chambers of the heart, the pericardium, or the interior of the cardiac muscle at the desired location. The cells may be administered to a recipient heart by intracoronary injection, e.g., into the coronary circulation. The cells may also be administered by intramuscular injection into the wall of the heart.

Medical indications for such treatment include treatment of acute and chronic heart conditions of various kinds, such as coronary heart disease, cardiomyopathy, endocarditis, congenital cardiovascular defects, and congestive heart failure. Efficacy of treatment can be monitored by clinically accepted criteria, such as reduction in area occupied by scar tissue or revascularization of scar tissue, and in the frequency and severity of angina; or an improvement in developed pressure, systolic pressure, end diastolic pressure, patient mobility, and quality of life.

The differentiating cells may be administered in any physiologically acceptable excipient, where the cells may find an appropriate site for regeneration and differentiation. The cells may be introduced by injection, catheter, or the like.

The cells of the disclosure can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types.

Cells of the disclosure may be genetically altered in order to introduce genes useful in the differentiated cardiomyocyte, e.g., repair of a genetic defect in an individual, selectable marker, etc. Cells may also be genetically modified to enhance survival, control proliferation, and the like. Cells may be genetically altering by transfection or transduction with a suitable vector, homologous recombination, or other appropriate technique, so that they express a gene of interest. The cells of the disclosure can also be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in cardiomyocytes. Many vectors useful for transferring exogenous genes into target mammalian cells are available. The vectors may be episomal, e.g. plasmids, virus derived vectors such cytomegalovirus. adenovirus, etc., or may be integrated into the target cell genome, through homologous recombination or random integration, e.g., retrovirus derived vectors such MMLV, HIV-1, ALV, etc. For modification of stem cells, lentiviral vectors are preferred. Lentiviral vectors such as those based on HIV or FIV gag sequences can be used to transfect non-dividing cells, such as the resting phase of human stem cells (see Uchida et al. (1998) P.N.A.S 95(20):11939-44).

In vitro cardiomyocytes and cardiac organoids produced by the methods of the disclosure also provide a source cells for novel cardiac drug discovery, development, and safety testing. Among the drugs that ultimately make it to market, many are later withdrawn due to side effects associated with electrophysiological alterations of the heart (Braam et al., 2010). The use of in vitro cardiomyocytes and cardiac organoids produced by the methods of the disclosure offers the pharmaceutical industry an invaluable tool for preclinical screening of candidate drugs to treat cardiomyopathy, arrhythmia, and heart failure, as well as therapeutics to combat secondary cardiac toxicities. The development of new screens using in vitro cardiomyocytes produced by the methods of the disclosure should reduce the time and cost of bringing new drugs to market.

In screening assays for biologically active agents (e.g., small molecule compounds, peptides, viruses, etc.) of the cardiomyocytes, usually a culture comprising the cardiomyocytes, is contacted with the agent of interest, and the effect of the agent assessed by monitoring output parameters, such as expression of markers, cell viability, electrophysiology, and the like.

Agents of interest for screening include known and unknown compounds that encompass numerous chemical classes, primarily organic molecules, which may include organometallic molecules, inorganic molecules, genetic sequences, etc. An important aspect of the disclosure is to evaluate candidate drugs, including toxicity testing; and the like.

In addition to complex biological agents, such as viruses, candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, hormones or hormone antagonists, etc. Exemplary of pharmaceutical agents suitable for this disclosure are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference.

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include manufacturing samples, pharmaceuticals, libraries of compounds prepared for analysis, and the like. Samples of interest include compounds being assessed for potential therapeutic value, i.e. drug candidates.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of cell samples, usually in conjunction with cells lacking the agent. The change in parameters in response to the agent is measured, and the result evaluated by comparison to reference cultures, e.g., in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus, preferred formulations consist essentially of a biologically active compound and a physiologically acceptable carrier, e.g., water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e., at zero concentration or below the level of detection of the agent or at or below the concentration of agent that does not give a detectable change in the phenotype.

The cells may be freshly isolated, cultured, genetically altered as described above, or the like. The cells may be environmentally induced variants of clonal cultures: e.g., split into independent cultures and grown under distinct conditions, for example with or without virus; in the presence or absence of other biological agents. The manner in which cells respond to an agent, particularly a pharmacologic agent, including the timing of responses, is an important reflection of the physiologic state of the cell.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g., mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

In vitro cardiomyocytes and cardiac organoids produced by the methods of the disclosure can also be used in developmental biology, disease modeling, and post-genomic personalized medicine. Deriving hiPSCs from patients with specific cardiac diseases, differentiating them to cardiomyocytes using the methods of the disclosure, and then performing electrophysiological and molecular analyses will provide a powerful tool for deciphering the molecular mechanisms of disease (Josowitz et al., 2011). Studies to date have largely concentrated on recapitulating genetic disease phenotypes in vitro, such as long QT syndromes (Itzhaki et al., 2011a; Matsa et al., 2011; Moretti et al., 2010), Timothy syndrome (Yazawa et al., 2011), and LEOPARD syndrome (Carvajal-Vergara et al., 2010). The possibility of modeling cardiac diseases without a known genetic element is another exciting prospect. The combination of novel drug discovery and efficacy testing with cardiomyocytes derived from patient-specific hiPSCs is a potentially groundbreaking option for personalized medicine.

### EMBODIMENTS

The following numbered embodiments also form part of the present disclosure:

1. A method of maintaining pluripotency of stem cells in three-dimensional (3D) culture, the method comprising: culturing pluripotent stem cells in suspension to form an aggregate in a medium comprising 5 μM or less of a Rock inhibitor.

2. The method of embodiment 1, wherein the medium comprises from about 0.01 μM to about 5 μM of the Rock inhibitor.

3. The method of embodiment 1 or embodiment 2, wherein the medium comprises from about 0.5 μM to about 2 μM of the Rock inhibitor.

4. The method of any one of embodiments 1-3, wherein the medium comprises about 1 μM of the Rock inhibitor.

5. The method of any one of embodiments 1-4, wherein the pluripotent stem cells are induced pluripotent stem cells.

6. The method of any one of embodiments 1-5, wherein the medium comprises a viscosity enhancer, optionally wherein the viscosity enhancer comprises methylcellulose.

7. The method of any one of embodiments 1-6, wherein the medium is mTeSR1 or StemFlex supplemented with the Rock inhibitor and methylcellulose.

8. The method of any one of embodiments 1-7, wherein prior to the culturing step, the pluripotent stem cells are cultured in a maintenance media on a substrate.

9. The method of embodiment 8, wherein the maintenance medium comprises DMEM/F12 supplemented with bFGF, TGF-β, γ-aminobutyric acid, LiCl, L-glutamine, MEM non-essential amino acids, NaHCO₃, chemically defined lipid concentrate, sodium selenite, bovine serum albumin, and β-mercaptoethanol.

10. The method of any one of embodiments 1-9, further comprising culturing the aggregate of pluripotent stem cells in suspension in a medium without Rock inhibitor.

11. The method of any one of embodiments 1-10, wherein a 3D pluripotent stem cell spheroid is produced.

12. The method of any one of embodiments 1-11, further comprising inducing differentiation of the pluripotent stem cells.

13. The method of embodiment 12, wherein the pluripotent stem cells are differentiated into ectoderm, mesoderm, or endoderm.

14. The method of embodiment 12 or embodiment 13, wherein the pluripotent stem cells are differentiated into cardiomyocytes.

15. A 3D pluripotent stem cell spheroid produced by the method of any one of embodiments 1-11.

16. A method of producing cardiomyocytes or a cardiac organoid, the method comprising: (a) culturing pluripotent stem cells in suspension to form an aggregate in a medium comprising 5 μM or less of a Rock inhibitor; (b) culturing the aggregate of pluripotent stem cells in suspension in a medium without Rock inhibitor; and (c) inducing cardiac differentiation of the aggregate of pluripotent stem cells, thereby producing the cardiomyocytes or a cardiac organoid.

17. The method of embodiment 16, wherein the medium of step (a) comprises from about 0.01 μM to about 5 μM of the Rock inhibitor.

18. The method of embodiment 16 or embodiment 17, wherein the medium of step (a) comprises from about 0.5 μM to about 2 μM of the Rock inhibitor.

19. The method of any one of embodiments 16-18, wherein the medium of step (a) comprises about 1 μM of the Rock inhibitor.

20. The method of any one of embodiments 16-19, wherein the pluripotent stem cells are induced pluripotent stem cells.

21. The method of any one of embodiments 16-20, wherein the medium of step (a) or the medium of step (b) comprises a viscosity enhancer, optionally wherein the viscosity enhancer comprises methylcellulose.

22. The method of any one of embodiments 16-21, wherein the medium of step (a) is mTeSR1 or StemFlex supplemented with the Rock inhibitor and methylcellulose.

23. The method of any one of embodiments 16-22, wherein prior to the culturing step, the pluripotent stem cells are cultured in a maintenance media on a substrate.

24. The method of embodiment 23, wherein the maintenance medium comprises DMEM/F12 supplemented with bFGF, TGF-β, γ-aminobutyric acid, LiCl, L-glutamine, MEM non-essential amino acids, NaHCO₃, chemically defined lipid concentrate, sodium selenite, bovine serum albumin, and β-mercaptoethanol.

25. The method of any one of embodiments 16-24, wherein inducing cardiac differentiation comprises culturing the aggregate of pluripotent stem cells in a medium comprising a Wnt signaling activator; and culturing the aggregate of pluripotent stem cells in a medium comprising a Wnt signaling inhibitor.

26. The method of embodiment 25, wherein the Wnt signaling activator comprises CHIR99021 and 6-bromoindirubin-3′-oxime (BIO).

27. The method of embodiment 25 or embodiment 26, wherein the Wnt signaling inhibitor comprises XAV939 and KY02111.

28. The method of any one of embodiments 16-27, wherein the outset beating time (OBT) is synchronized within about 24 hours.

29. The cardiomyocytes or cardiac organoid produced by the method of any one of embodiments 16-28.

30. A method for screening an agent for improving or diminishing cardiac function, the method comprising: contacting the cardiomyocytes or cardiac organoid of embodiment 29 with the agent; and measuring at least one response of the cardiomyocytes or cardiac organoid.

31. A therapeutic agent comprising the cardiomyocytes or cardiac organoid of embodiment 29.

32. A method for treating a disorder of a cardiac tissue, the method comprising: transplanting the cardiomyocytes or cardiac organoid of embodiment 29 into a heart of a subject in need of treatment.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

### Example 1: Unprecedented Capacity of 3D Cardiac Differentiation of Human eiPSCs Cultured in 3D with Low RI

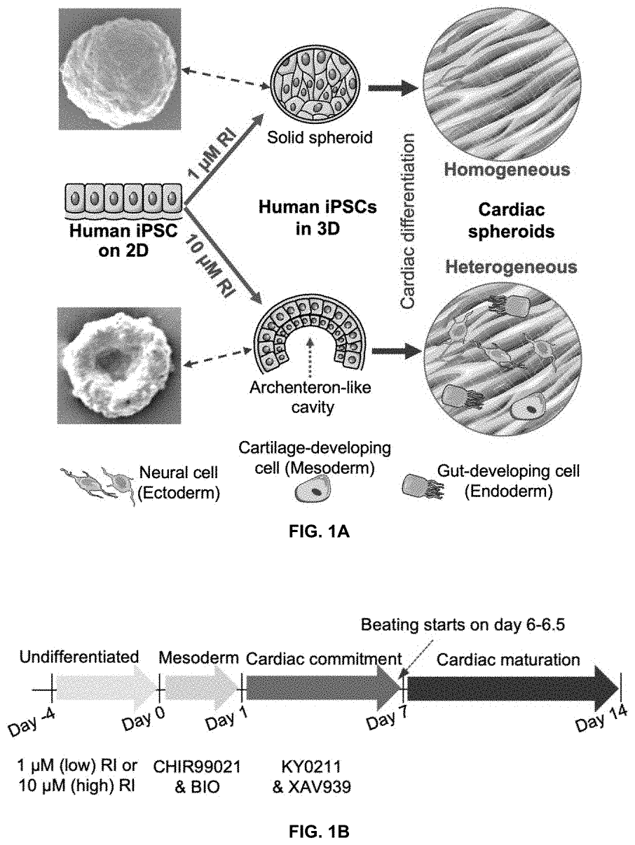

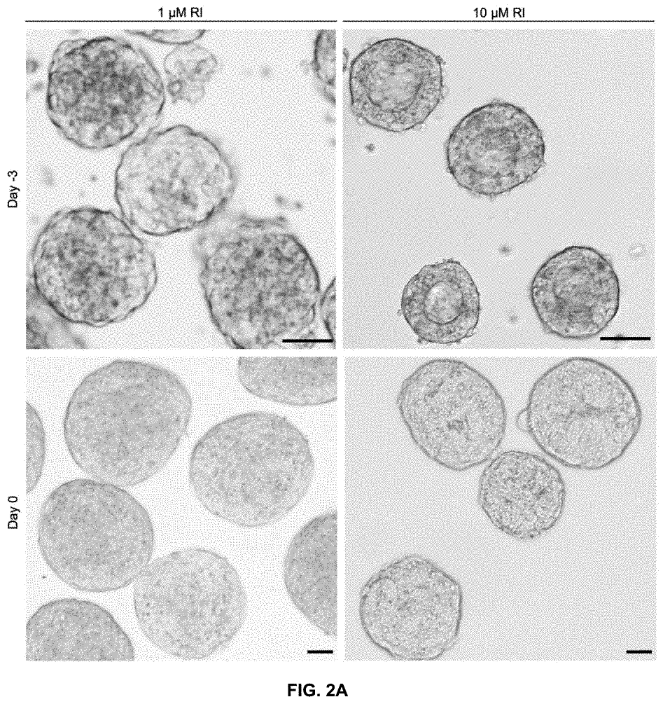

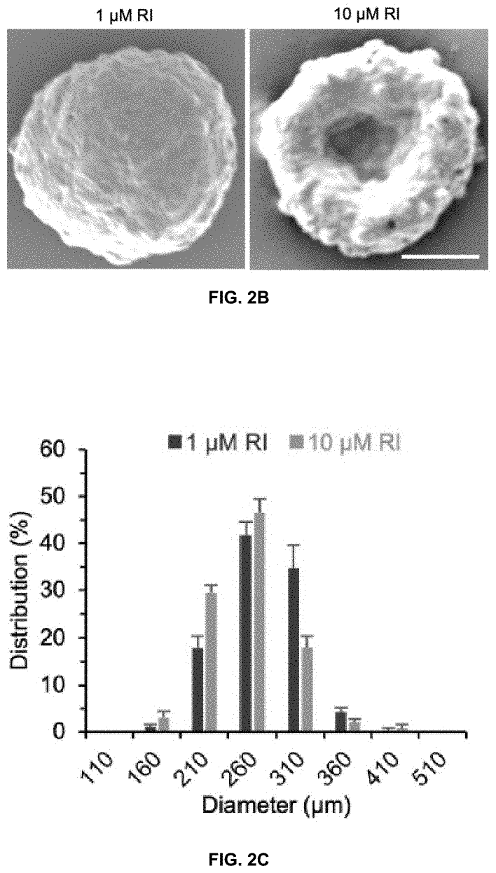

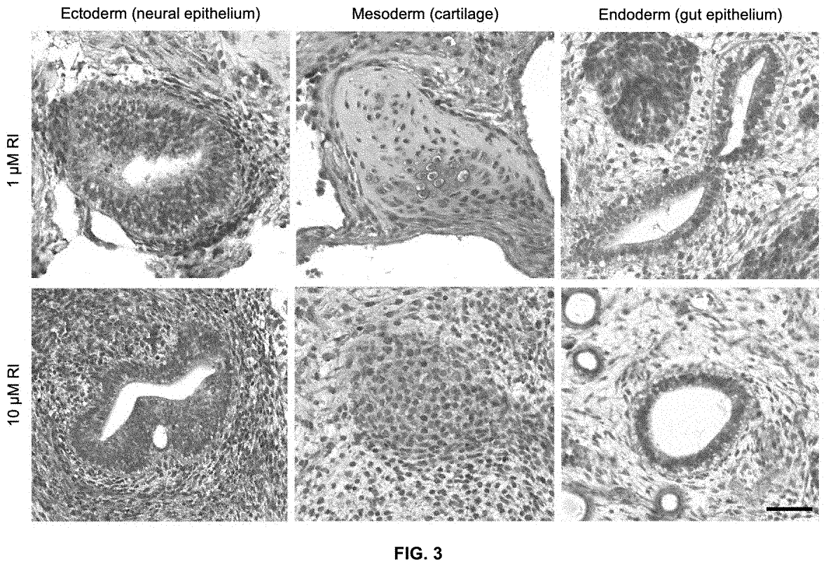

The timeline for the 4-day (from day −4 to 0) 3D culture and the subsequent 3D cardiac differentiation of iPSCs is illustrated in FIG. 1B. First, 3D eiPSC spheroids were obtained for 3D cardiac differentiation, for which the eiPSC colonies from 2D culture were detached and mechanically cut with a cell strainer (100 μm mesh size) into small iPSC clumps for suspension culture in the mTeSR medium for 4 days. Methylcellulose was added into the medium to enhance the medium viscosity and reduce fusion of the eiPSC clumps/spheroids during the suspension culture. During the first two days of 3D culture, the medium was supplemented with 1 μM RI (low RI) or 10 μM RI (high RI that has been commonly used for improving the survival/yield of human PSCs including iPSCs during culture), and no RI was used during the last two days of culture. Typical images showing the morphology of the iPSC spheroids under suspension culture on days −3 and 0 from both the 1 and 10 μM RI groups are shown in FIG. 2A-B. The size (in diameter) distributions of the eiPSC spheroids on day 0 for both groups are given in FIG. 2C: The spheroids were 245.3±42.0 μm and 227.9±38.5 μm and there were 3172±272 and 2662±207 cells per spheroid, for the 1 and 10 μM RI groups, respectively. The pluripotent nature of the eiPSC spheroids from both groups were confirmed by their capability of forming teratomas consisting of tissues from all the three germ layers (i.e., neural epithelium, cartilage, and gut epithelium for ectoderm, mesoderm, and endoderm, respectively, FIG. 3).

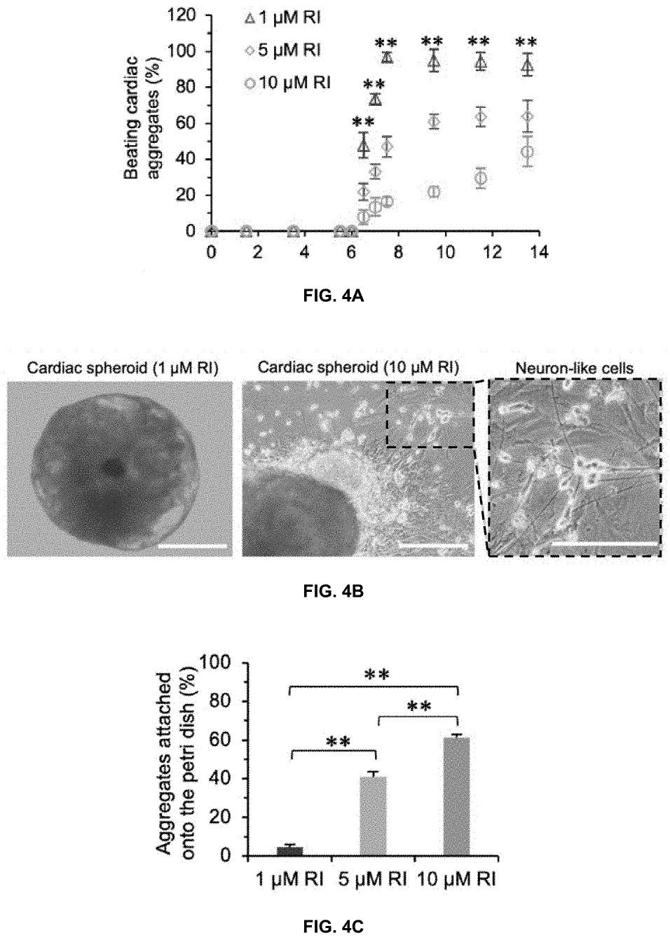

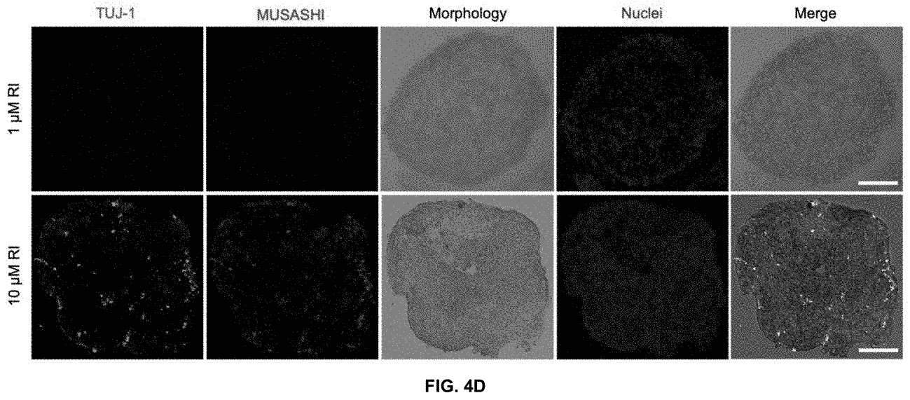

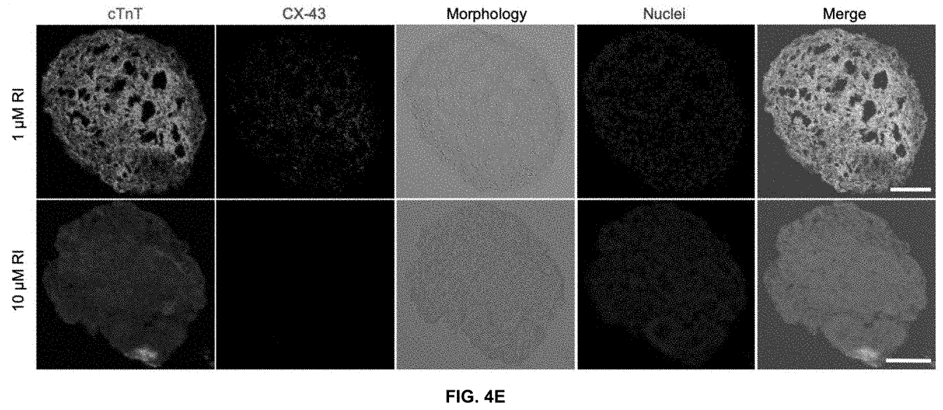

Cardiac differentiation of the 3D eiPSC spheroids was conducted by modulating the canonical Wnt signaling pathway with agonists and antagonists sequentially, which has also been used for differentiation of human PSCs into cardiomyocytes under 2D culture. Two Wnt agonists, CHIR99021 (8 μM) and 6-bromoindirubin-3′-oxime (BIO, 2 μM), were supplemented into the culture medium on day 0, to up-regulate the Wnt signaling and induce iPSC differentiation into mesoderm for 1 day. On day 1, two Wnt antagonists, XAV939 (10 μM) and KY02111 (10 μM), were used to suppress the Wnt signaling for inducing cardiac commitment of the mesoderm cells in the following 6 days to obtain cardiac spheroids. Afterward, the cardiac spheroids were cultured in pure cardiac maintenance medium for maturation until day 13.5. Spontaneous beating was observed for ˜48% of the cardiac spheroids in the 1 μM RI group on as early as day 6.5, and the percentage of beating cardiac spheroids peaked at ˜97% at ˜24 h later on day 7.5 (FIG. 4A). In other words, the outset beating time (OBT) of the cardiac spheroids was synchronized to be within ˜24 h for the 1 μM RI group. No significant change in the beating percentage from day 7.5 to day 13.5 was observed. In contrast, in the 10 μM RI group, only ˜8% and ˜44% of cardiac spheroids beat on days 6.5 and 13.5, respectively (FIG. 4A, on day 13.5). Moreover, the 5 μM RI group showed a shortened OBT of 3 days (from day 6.5 to day 9.5) and ˜61% beating cardiac spheroids on days 9.5 (FIG. 4A). Therefore, the OBT ranges over at least a week, indicating high heterogeneity of the cardiac spheroids in the 10 μM RI group as the beating is a direct and visible indicator of functional maturity of the cardiac spheroids. In other words, the 3D cardiac differentiation of eiPSCs in the 5 and 10 μM group is not only significantly less efficient (and lengthier) but also less homogeneous than that in the 1 μM RI group.

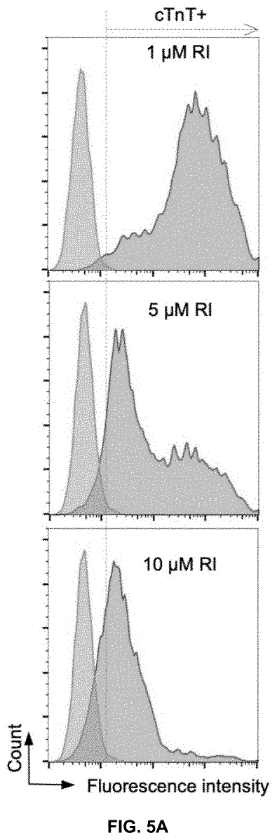

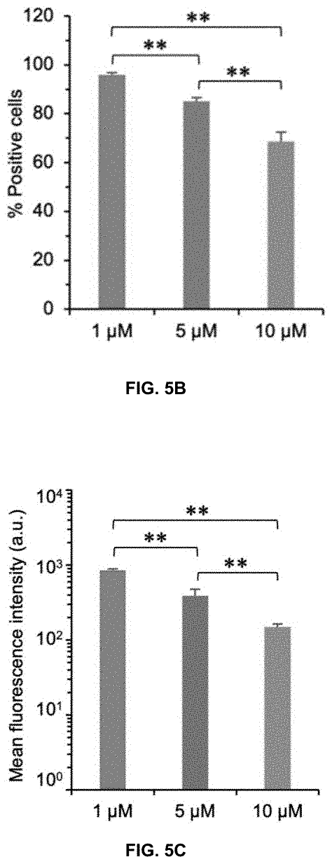

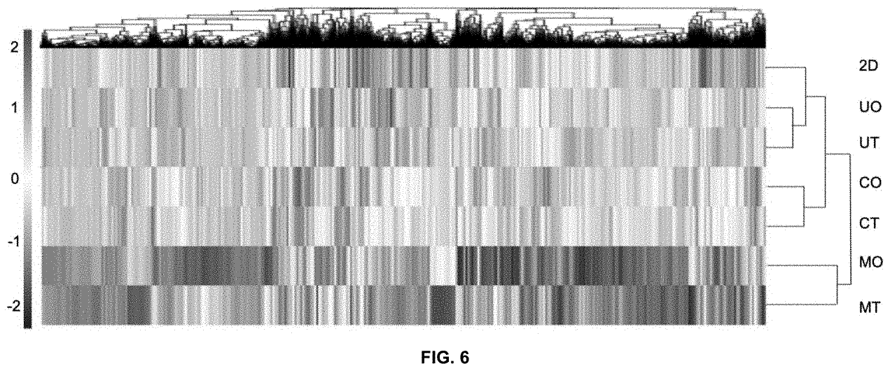

To further investigate the difference of cells in cardiac spheroids between the 1 and 10 μM RI groups, the cardiac spheroids on day 13.5 from both groups were re-plated in regular cell culture petri dish. After 3 days, ˜61% of the cardiac spheroids in the 10 μM RI group attached to the 2D surface, and some of the cells migrated out of the spheroids appeared to be neuron-like with neurite-like processes (FIG. 4B-C). In contrast, less than 5% of the cardiac spheroids in the 1 μM RI group became attached after the re-plating and migration of cells out of the cardiac spheroids was negligible, while the 5 μM RI group showed ˜41% attached cardiac spheroids (FIG. 4B-C). Moreover, both the percentage of cTnT-positive cells in the cardiac spheroids and the mean intensity of cTnT fluorescence staining (representing the cTnT protein expression in the cardiac spheroids) decreased with the increase of the RI concentration from 1 to 10 μM RI (FIG. 5), showing high concentration of RI compromises the cardiac differentiation of the human eiPSCs. To confirm the aforementioned observation of neuron-like cells in the 10 μM RI group, immunofluorescence staining of cryosectioned cardiac spheroids on day 12.5 in the 1 and 10 μM RI groups was conducted. Indeed, cells that were positive for neural lineage markers TUJ-1 and MUSASHI-1 were observed in the cardiac spheroids from the 10 μM RI group (FIG. 4D). In stark contrast, no evident staining of MUSASHI-1 and TUJ-1 was observable in the cardiac spheroids from the 1 μM RI group. Furthermore, the cardiac sarcomere-specific protein cTnT and gap junction (for conduction of cardiac potential) protein CONNEXIN-43 (CX-43) were highly expressed in the cardiac spheroids of the 1 μM RI group (FIG. 4E), while their expression was either weak (for cTnT) or barely visible (for CX-43) in the cardiac spheroids of the 10 μM RI group. Taken together, these data show that the 10 μM RI compromises cardiac differentiation of eiPSCs in 3D with a noticeable existence of neuron-like cells, and by reducing the RI to 1 μM, a protocol is developed to achieve highly efficient and synchronized cardiac differentiation of the eiPSCs in 3D. Therefore, studies were performed to further understand the mechanisms of the improved cardiac differentiation with the reduced RI.

### Example 2: High RI-Induced Heterogeneous Gene Expression of eiPSCs Under 3D Culture to Compromise their 3D Cardiac Differentiation

To investigate the mechanisms for the different outcomes when using different RI concentrations for culturing the eiPSC spheroids in 3D for cardiac differentiation, samples at various stages of the 3D culture and differentiation shown in FIG. 1 were collected and analyzed by RNA-sequencing (RNA-seq). The sample groups and their abbreviations are summarized in Table 1 including undifferentiated eiPSCs on day −2.5 (U), cardiac commitment on day 2.5 (C), cardiac maturation on day 13.5 (M), together with 2D monolayer colonies on day −4 (2D) as a common 2D undifferentiated control for both the 1 (one: 0) and 10 (ten: T) μM RI 3D culture. The raw data of RAN-seq were of high quality with an error rate of no more than 0.03%. The raw data passed quality control were then aligned for mapping to the reference (human genome) with a total mapping rate of 95.8±0.5%. Afterward, the gene expression level was calculated by the number of mapped reads, also known as fragments per kilobase of transcript per million mapped reads (FPKM). Moreover, the global transcriptome of different samples with cluster analysis and hierarchical clustering analysis was carried out with log₁₀ (FPKM+1) on differentially expressed genes within all comparison groups and plotted as a heat-map (FIG. 6), showing the global gene expression pattern of samples together with the samples' hierarchies that match with the different stages of the different samples given in FIG. 1.

TABLE 1

| RI | Undifferen- | Cardiac | Cardiac | 2D control |
|---|---|---|---|---|
| dosage | tiated (U) | Commitment (C) | Maturation (M) | (2D) |
| 1 μM | UO | CO | MO | 2D |
| (One, O) | | | | |
| 10 μM | UT | CT | MT | |
| (Ten, T) | | | | |

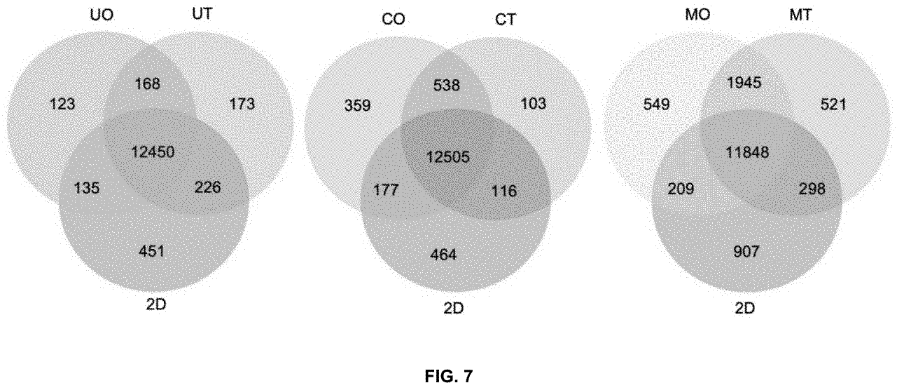

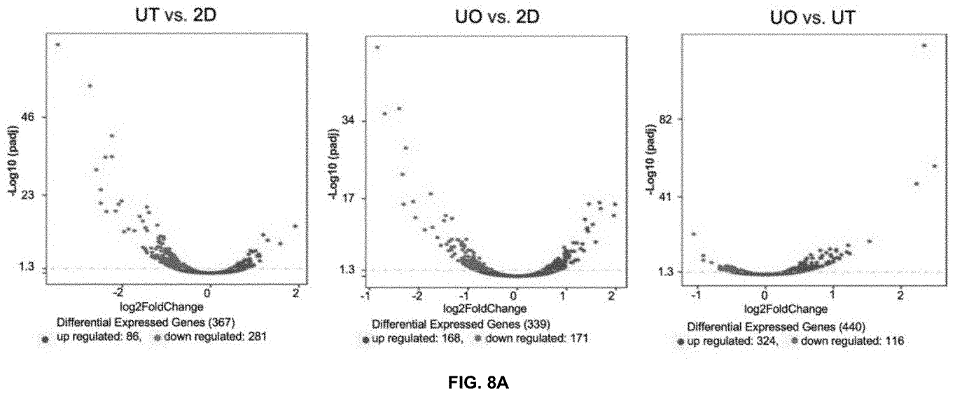

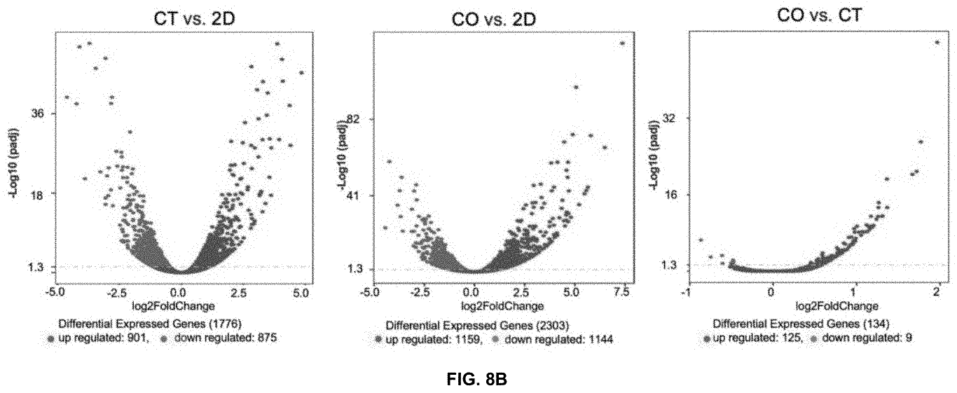

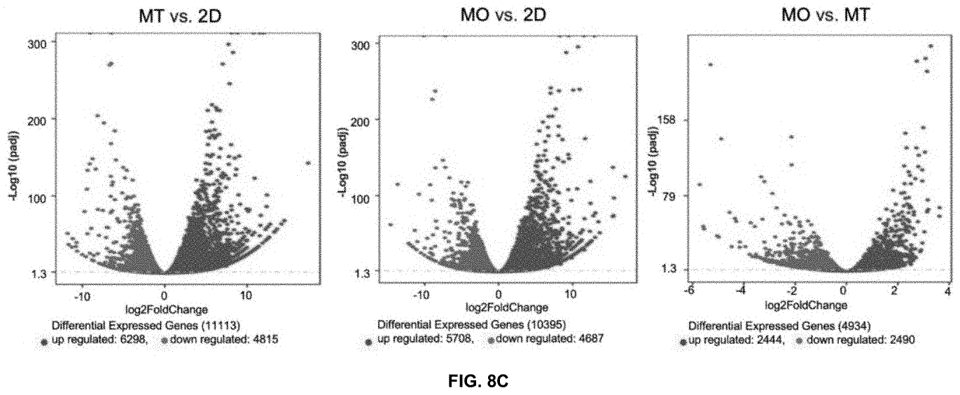

To understand the number of genes that are uniquely and co-expressed within the cells of the 1 versus 10 μM RI group at different stages of 3D culture and cardiac differentiation as compared to the 2D control, the Venn diagrams were generated first and shown in FIG. 7. The total number of co-expressed genes were similar in three groups at all three stages (UO/UT/2D: 12450, CO/CT/2D: 12505, and MO/MT/2D: 11848), but the differentially expressed unique genes increased from undifferentiated stage to cardiac commitment and then cardiac maturation (UO/UT/2D: 123/173/451, CO/CT/2D: 359/103/464, and MO/MT/2D: 549/521/907). This was further shown in the Volcano plots of the overall distribution of differentially expressed genes with a threshold being less than 0.05 for the adjusted p value (padj) (FIG. 8). This trend of increased significantly differentially expressed genes with the advance of the stage of differentiation was due to the increased expression of somatic genes from the early to late stages of cardiac differentiation.

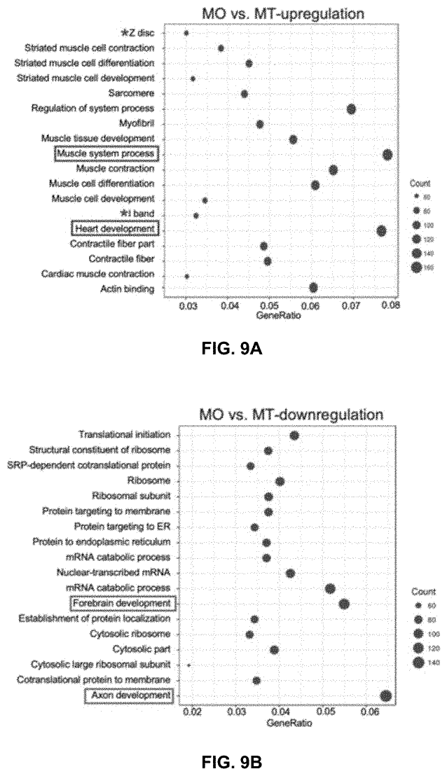

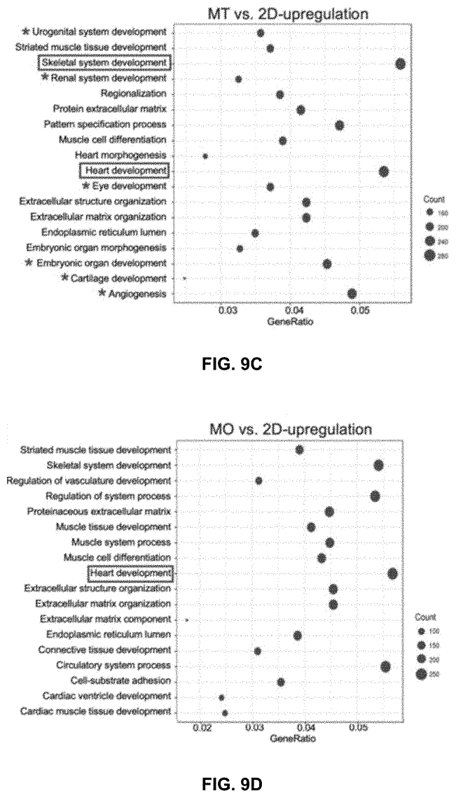

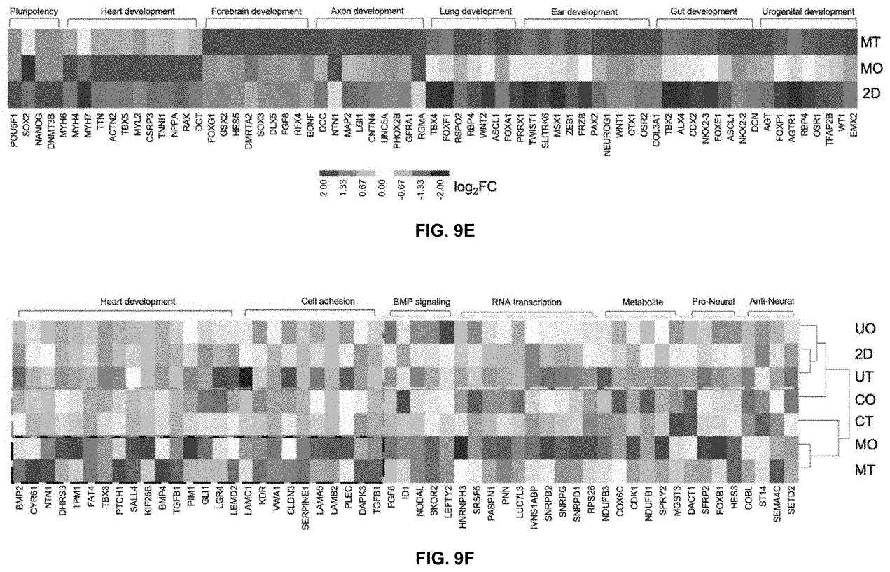

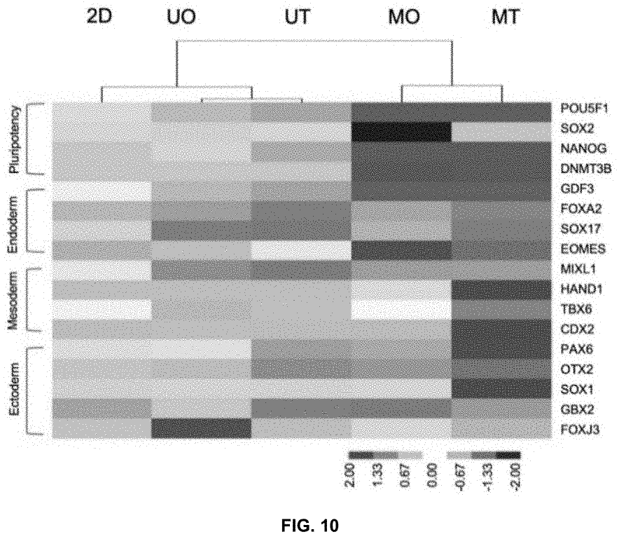

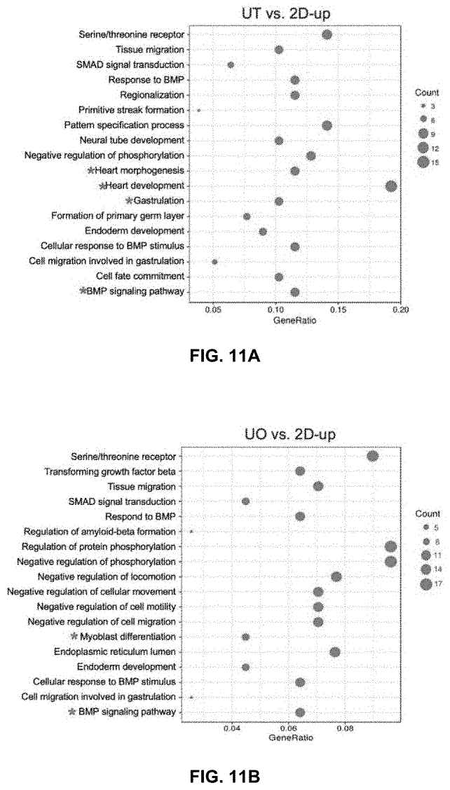

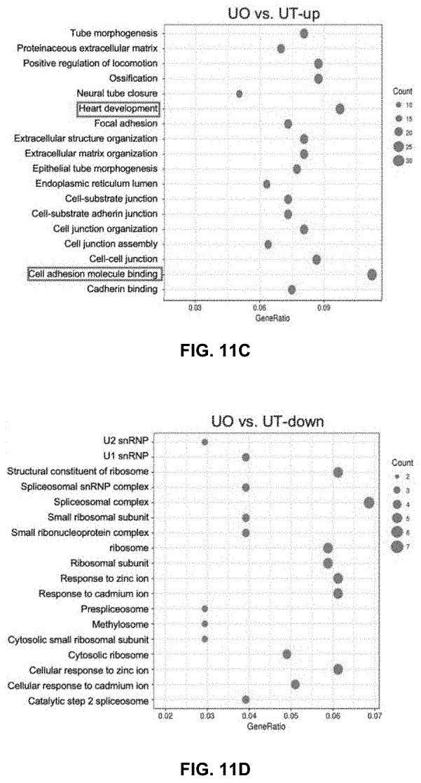

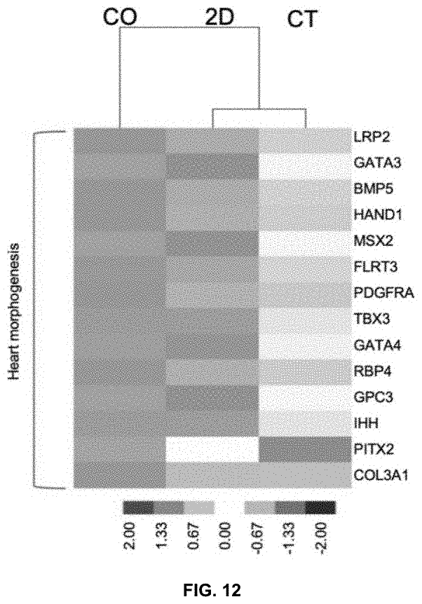

The differentially expressed genes were further examined through an enrichment analysis with the Gene Ontology (GO) database as the reference, to determine the biological functions or pathways significantly enriched with the genes. Notably, the top 18 significantly upregulated differentially expressed gene groups of MO with regard to MT (MO vs. MT) were enriched to heart development, muscle system process, and the development of Z-disc and I band (FIG. 9A), suggesting that the cardiomyocytes in the MO group had improved development/maturation in sarcomere organization compared to the cardiomyocytes in the MT group. The top 2 significantly down-regulated differentially expressed gene groups of MO vs. MT were enriched to the forebrain and axon development of the ectoderm-derived lineages (FIG. 9B). As expected, the cardiomyocytes in both MO and MT groups show a prominent upregulation of differentially expressed gene groups enriched to heart development compared to the control 2D group (MT vs. 2D: top 2 in FIG. 9C and MO vs. 2D: top 1 in FIG. 9D). Notably, MT has many differentially expressed genes enriched to heterogeneous endoderm (e.g., embryonic development of organs like lung and gut) and non-cardiac mesoderm (e.g., cartilage and urogenital) tissue development, in addition to the ectoderm tissue (e.g., eye) development (FIG. 9C-D), which was not observed for MO (FIG. 9D). The enriched functional clusters with detailed genes were plotted in a heat-map in addition to the expression of pluripotent genes (e.g., POU5F1, SOX2, NANOG, and DNMT3B) that were all decreased in MO and MT (FIG. 9E and FIG. 10). Importantly, SOX2 which is important for neural stem cell development has higher expression in MT than MO, which indicates an increased shift toward the neural lineage of cells in the MT group compared to the MO group. Interestingly, the early cardiac-associated genes (e.g., SALL4, NTN1, and GLI1) were expressed as early as the undifferentiated stage (U) when the pluripotency genes were dominant in the transcriptome (FIG. 9F, FIG. 10, FIG. 11A-B). There were upregulated genes enriched to heart development in UO compared to UT (FIG. 11C). Furthermore, genes (e.g., FGF8, ID1, NODAL, SKOR2, and LEFTY2) enriched to the BMP signal pathway involving in cardiogenesis, were upregulated in UO compared to UT (FIG. 9F, FIG. 11C). In addition, upregulated genes enriched to cell adhesion were also present in UO compared to UT (FIG. 11C). However, the genes enriched to RNA transcription and metabolism were down-regulated in UO but up-regulated in UT compared to 2D (FIG. 9F, FIG. 11D). Moreover, there were neural tube closure associated genes (e.g., COBL, ST14, SEMA4C, and SETD2) upregulated in UO compared to UT, while pro-neural development genes (e.g., DACT1, SFRP2, FOXB1, and HES3) were upregulated in UT compared to UO (FIG. 9F). At the intermediate CO/CT stage, it shows significantly upregulated expression of genes (e.g., LGR4, LRP2, GATA3, BMP5, HAND1, MSX2, and PDGFRA) enriched to heart development in the CO group compared to the CT group (FIG. 9F, FIG. 12).

### Example 3: High RI-Induced Heterogeneous Protein Expression of eiPSCs Under 3D Culture to Compromise their 3D Cardiac Differentiation

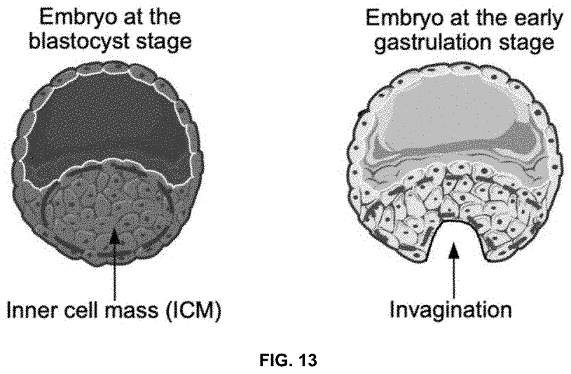

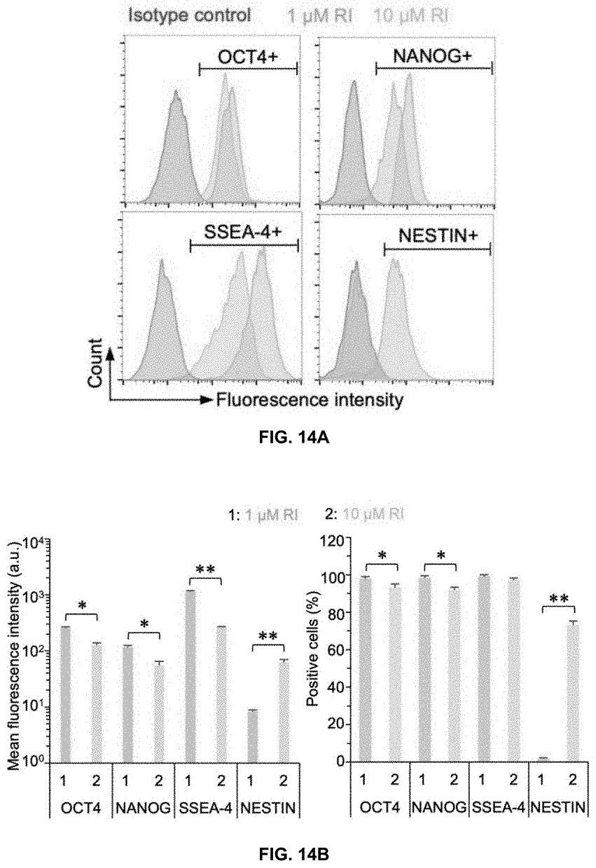

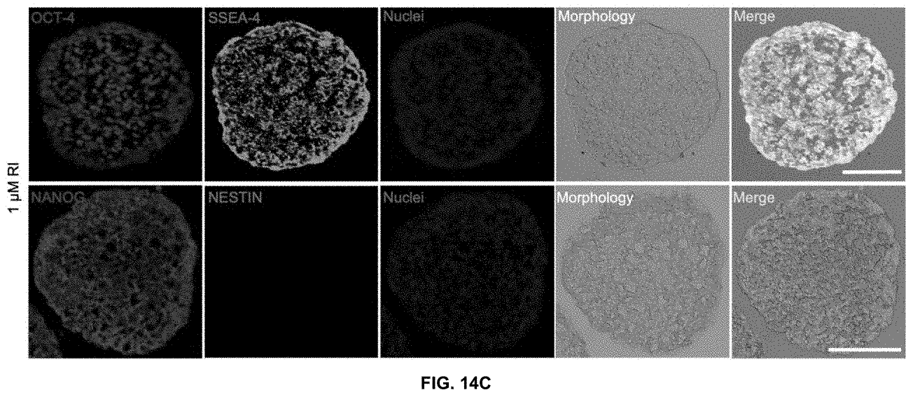

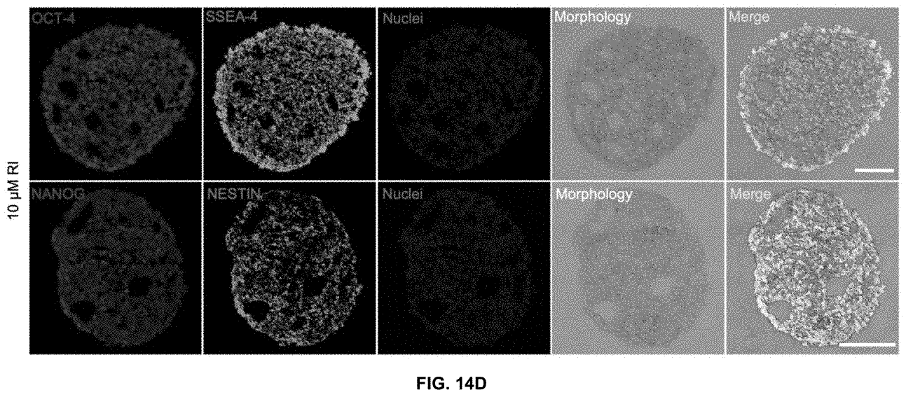

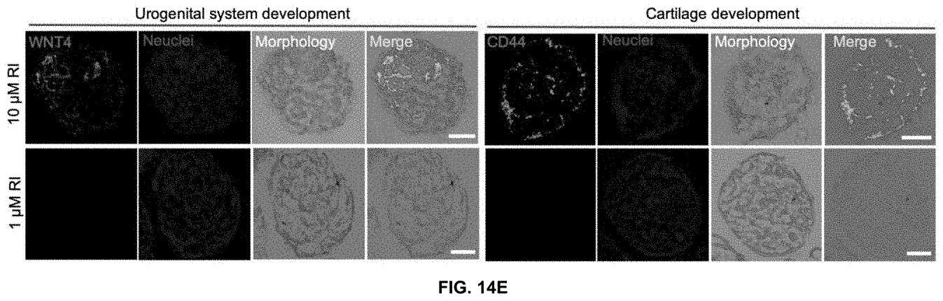

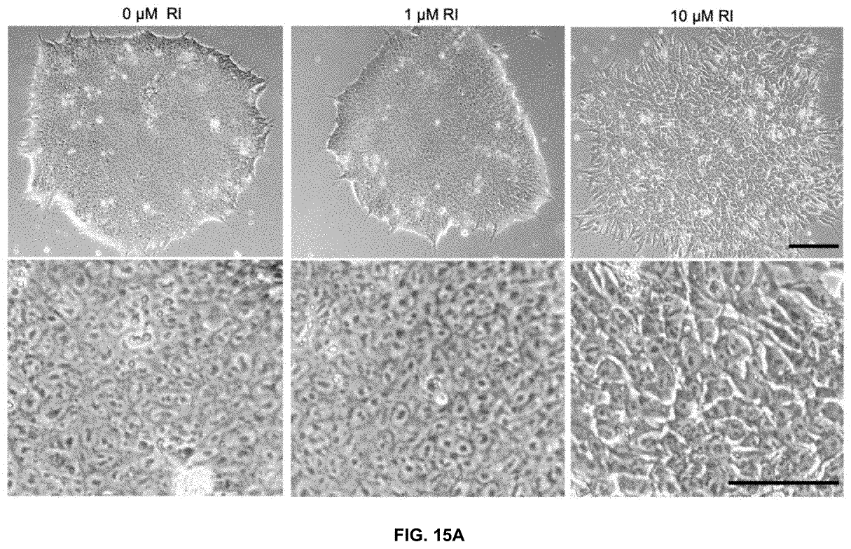

The heterogeneous protein expression induced by high RI was first indicated by the morphology of the eiPSC spheroids (FIG. 2A-B): nearly all the eiPSC spheroids after one-day culture on day −3 in the 10 μM RI group had an evident core-shell morphology with a cavity-like core and cells being in a shell (although it becomes less evident on day 0, probably due to the inward growth of the cells in the shell). This was not observable for the iPSC spheroids that remain solid-like in the 1 μM RI group, similar to the solid-like inner cell mass that contains pluripotent (i.e., embryonic) stem cells in the early stage of blastula (FIG. 13). This structure of cell aggregates with an archenteron-like cavity (FIG. 2A-B) for the 10 μM RI group resembles that of the epiblast and hypoblast-like cells (differentiated from the solid inner cell mass after invagination during early gastrulation). The aforementioned morphological difference of the eiPSC spheroids treated with 1 μM RI and 10 μM RI on day −3 was further confirmed with SEM imaging (FIG. 2B). Hence, we examined the expression of pluripotency and ectoderm differentiation protein markers within the eiPSC spheroids after four-day culture from the 1 and 10 μM RI groups using flow cytometry. The expression of three pluripotency markers including OCT-4, NANOG, and SSEA-4 was significantly higher in the 1 μM RI group, judged by the median fluorescence intensity and percentage of positive cells for the pluripotency markers (FIG. 14A-B). Furthermore, the expression of the ectoderm maker NESTIN was high (73.1%) in the 10 μM RI group. In stark contrast, the expression of NESTIN was negligible in the 1 μM RI group. This was further confirmed by immunostaining of cryo-sectioned slices of the eiPSC spheroids with the various pluripotency and ectoderm markers (FIG. 14C-D). This unexpected ectoderm induction of human iPSCs by the 10 μM RI under 3D culture compromises their capability of differentiating into cells of non-ectoderm origin including cardiomyocytes.

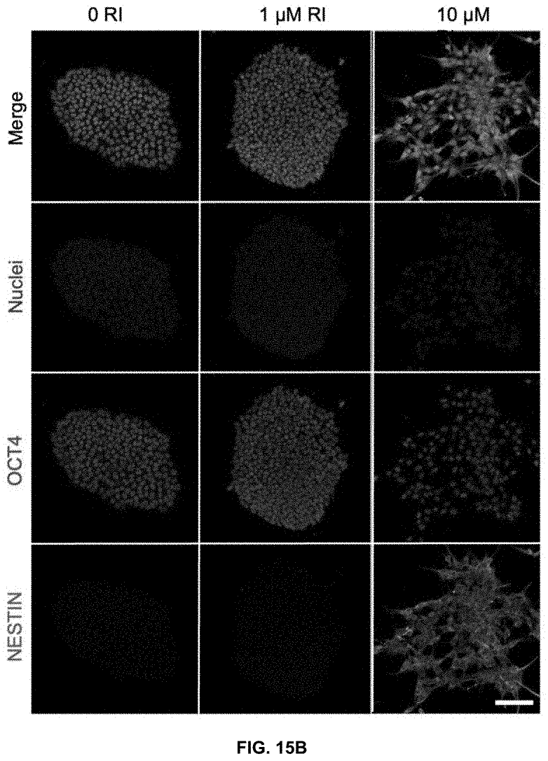

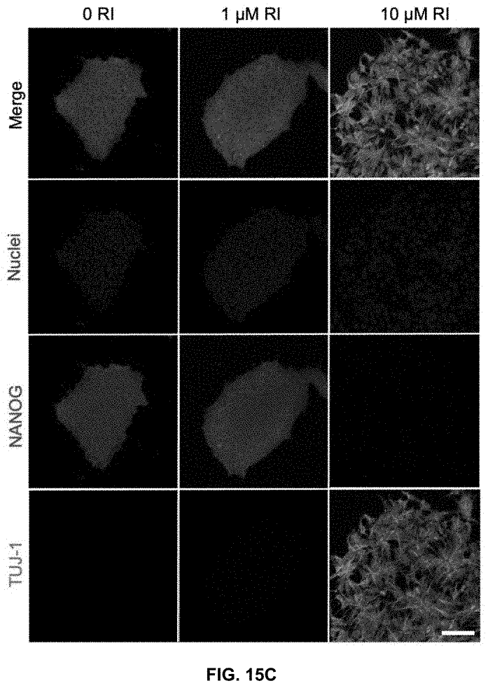

To further confirm the aforementioned difference in morphology and protein expression between the 1 and 10 μM RI groups, the 3D iPSC spheroids obtained with 0, 1, and 10 μM RI concentrations collected on day 0 (i.e., after 4-day culture in 3D) were plated on the 2D surface coated with Matrigel in Petri dish (i.e., under conventional 2D culture) and cultured for 2 days to observe the cell morphology. Although eiPSC colonies were observable for all the three groups, the ones in the 0 and 1 μM RI groups were typical with tightly packed cells consisting mainly of nuclei inside them and a largely smooth outer boundary (FIG. 15A). In contrast, the cells in the eiPSC colonies of the 10 μM RI group were more loosely packed with a reduced ratio of nuclei to the cytoplasm in volume and the colonies had a largely spiky outer boundary with sprawled-out differentiated cells (FIG. 15A), suggesting spontaneous differentiation of the eiPSCs in the 10 μM RI group during culture. This was confirmed by immunostaining of the iPSCs spheroids: positive staining of neural markers (NESTIN and TUJ-1) and weakened/no staining of pluripotency markers (OCT-4 and NANOG) were observable in the colonies of the 10 μM RI group; in contrast, the colonies of the 0 and 1 μM RI groups show positive expression of OCT-4 and NANOG and were negative for NESTIN and TUJ-1 (FIG. 15B-C).

Lastly and importantly, protein markers for urogenital system development (WNT4) and cartilage development (CD44) are also observable in the eiPSC-derived cardiac spheroids of the MT group but not the MO group (FIG. 14E), confirming at the protein expression level, the heterogeneous non-cardiac mesoderm tissue development in the MT (but not MO) group identified earlier by the RNA-seq gene analysis (FIG. 9C). Collectively, the commonly used high concentration (10 μM) RI causes heterogeneous differentiation of eiPSCs under 3D culture and adversely compromises their cardiac differentiation in 3D, which may be overcome by reducing the RI concentration to 1 μM. Therefore, the quality of cardiac differentiation of the eiPSC spheroids from the low (1 μM) RI group was further studied.

### Example 4: Characterization of High-Quality Cardiac Differentiation of iPSCs in 3D with Low RI

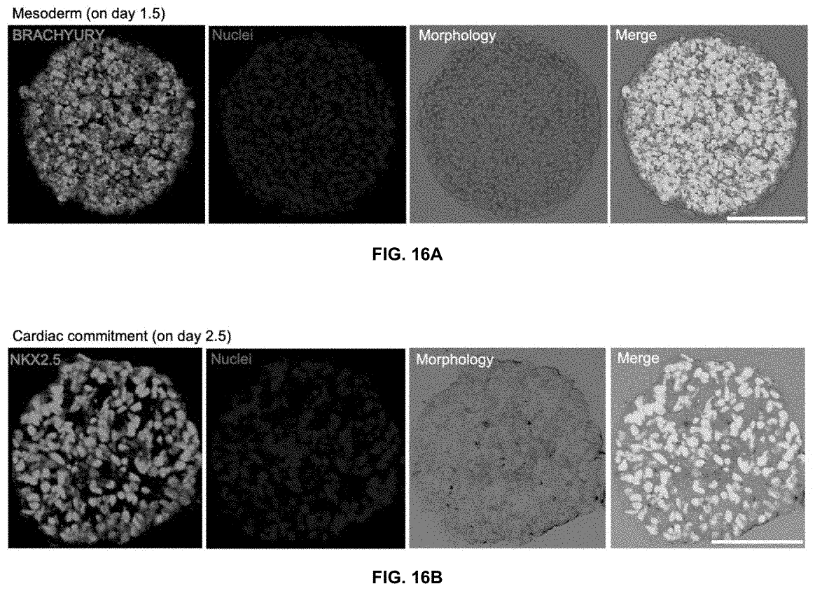

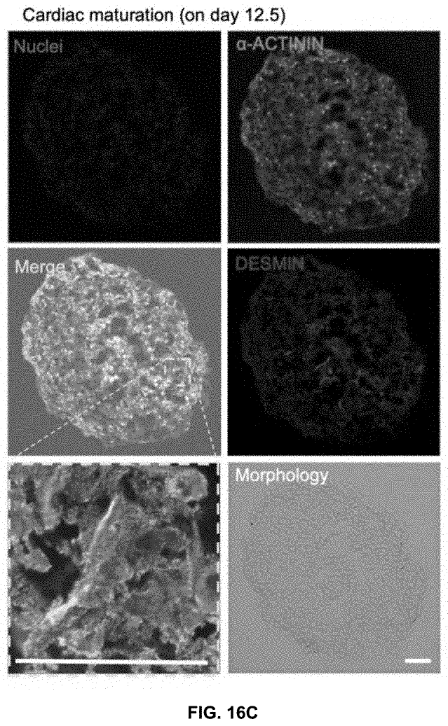

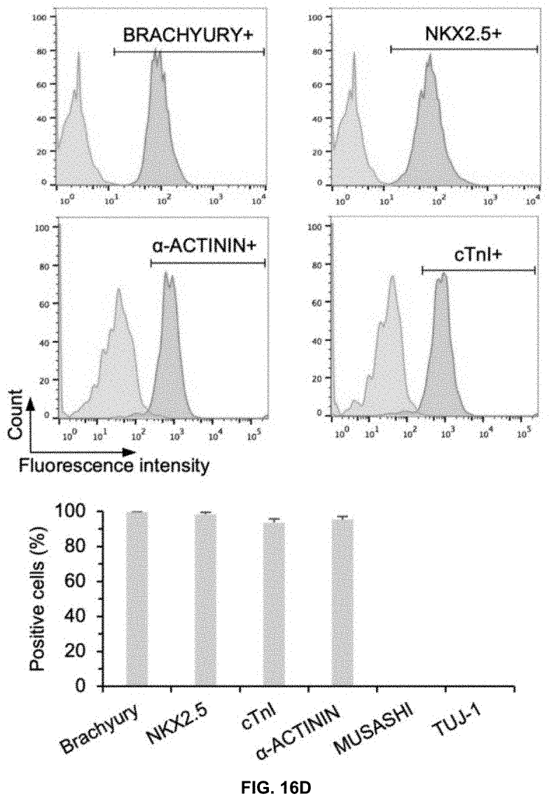

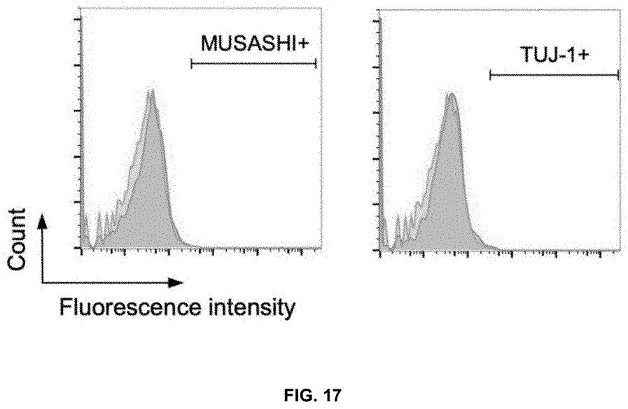

Probably due to the high and homogeneous pluripotency of the eiPSC spheroids in the low RI group according to the aforementioned transcriptomic and protein analyses, the cardiac differentiation procedure results in high purity of cells at each of the three stages of modulating the Wnt signal pathway (FIG. 16A-D). After incubation with agonists of Wnt signaling (CHIR99021 and BIO) for 1 day, the eiPSCs were successfully induced into the mesoderm lineage. This was confirmed by the highly and homogeneously positive staining for the mesoderm protein marker BRACHYURY in the spheroids (FIG. 16A) and the ˜100% BRACHYURY-positive cells in the spheroids according to flow cytometry analysis (FIG. 16D). Afterward, the mesoderm cells were induced with antagonists of Wnt signaling (XAV-939 and KY02111) for two days to commit to the cardiac lineage on day 3, which was confirmed with the homogeneous (FIG. 16B) and high (˜100%, FIG. 16D) expression of the cardiac progenitor cell protein marker NKX2.5. After further cardiac commitments to day 6 and cardiac maturation to day 12.5, the cardiac spheroids had abundant sarcomeres with a homogeneous expression of myofibril-associated protein α-ACTININ and intermediate filament protein DESMIN, showing advanced development of sarcomere organization (FIG. 16C). This was further confirmed by quantitative analyses of the sarcomere-related proteins including α-ACTININ and cTnI, which shows more than 90% of the cells in the cardiac spheroids on day 12.5 were positive for all the three protein markers (FIG. 16D). In contrast, cells positive for the neural lineage cell makers MUSASHI-1 and TUJ-1 were negligible (FIG. 16D, FIG. 17), indicating a high-quality cardiac differentiation via the reduction of RI concentration for culturing the eiPSCs in 3D before initiating cardiac differentiation to minimize their heterogeneous differentiation.

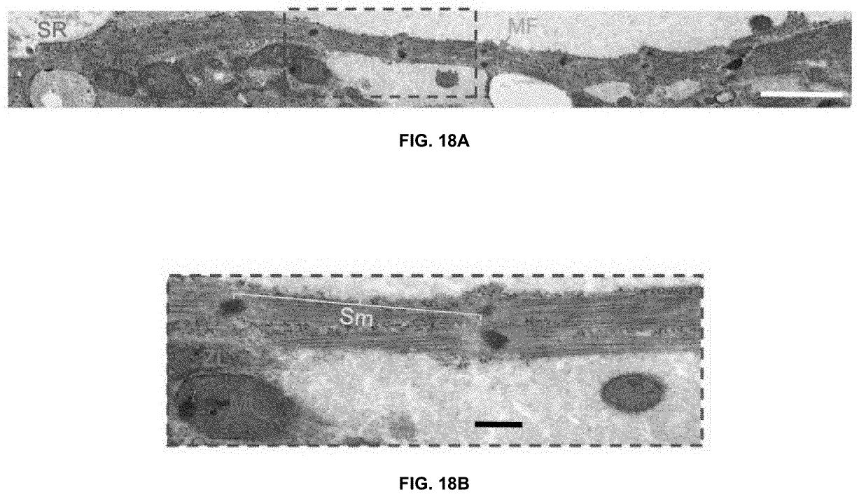

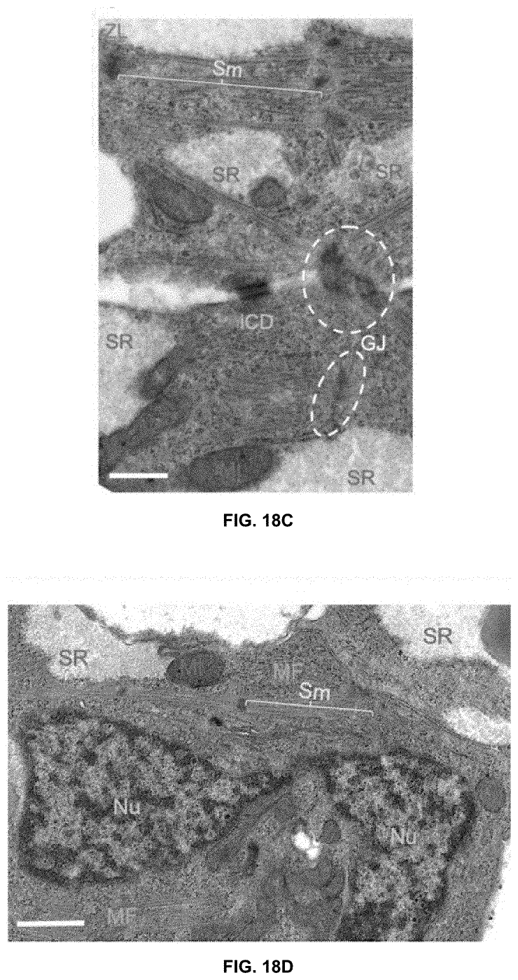

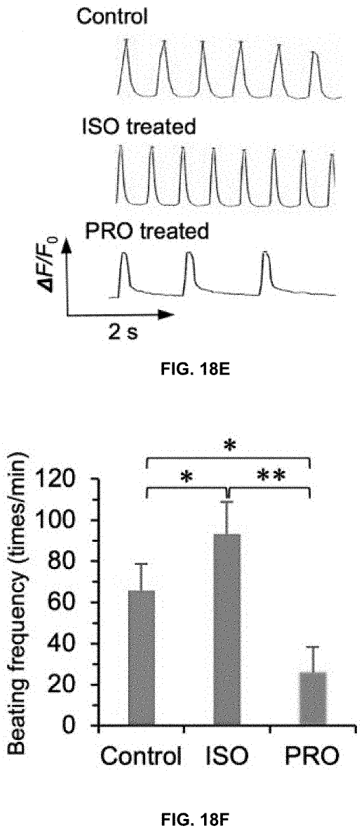

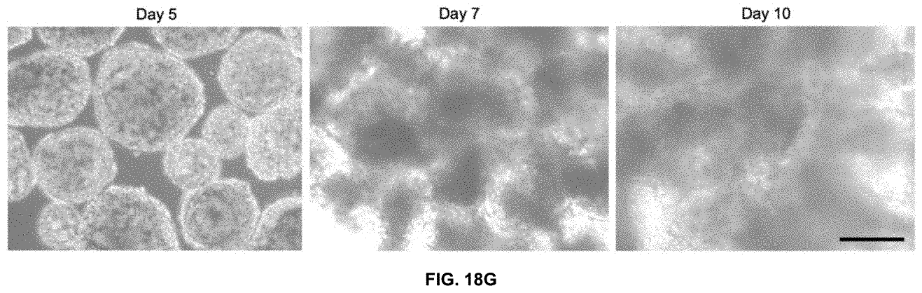

The quality of the cardiac spheroids in the low RI group on day 12.5 was further analyzed with transmission electron microscopy (TEM) to identify the key cardiomyocyte functional ultrastructures. The cardiomyocytes in the cardiac spheroids developed plenty of myofibrils (MF, up to 18 μm) and sarcoplasmic reticula (SR), which were typical functional components of cardiac muscle (FIG. 18A). Importantly, there were matured sarcomeres (Sm) between two Z lines (ZL, or Z discs) with an average length of 1.7±0.1 μm and width of 0.6±0.1 μm (FIG. 18B) inside the myofibril. The abundant mitochondria and sarcoplasmic reticula (SR) support the contractile function of the sarcomeres (FIG. 18A-C) by providing energy and calcium, respectively. There were also plenty of gap junctions and intercalated discs (iCD) located between the cell-cell membranes, which suggests a matured state of these ultrafine structures for signal transduction between cardiomyocytes in the cardiac spheroids (FIG. 18C). Moreover, there were cardiomyocytes with multiple nuclei (Nu) per cell, which indicates the maturation of cardiomyocytes (FIG. 18D). Collectively, the TEM data indicate critical ultrastructural evidence for the successful differentiation and maturation of cardiomyocytes as early as day 12.5 post-cardiac differentiation.

A calcium spike assay was also conducted with the cardiac spheroids on day 12.5 from the low HR group to examine their beating function, for which the fluo-4 staining was used to visualize the calcium transient in the cardiac spheroids. Furthermore, cardiac drugs isoproterenol (ISO) that speeds up beating and propranolol (PRO) that slows down beating were used to test the drug response of the cardiac spheroids (FIG. 18E-F). Before the drug treatments, the cardiac spheroids maintained a beating rate of 65±13 beats per minute, similar to that of a healthy adult human heart. When treated with ISO, the beating rate was significantly increased to 93±15 beats per minute. The beating rate was subsequently decreased to 26±12 beats per minute after treating the cardiac spheroids with PRO. Overall, this data shows that the cardiac spheroids derived from eiPSCs with reduced RI concentration develop normal beating activities and may serve as an in vitro model for cardiac research and drug screening.

It is worth noting that the human eiPSC-derived homogeneous cardiac spheroids were compatible with GelMA that has been widely used for 3D bioprinting. The cardiac spheroids collected on day 5 post-cardiac differentiation (before the initiation of spontaneous beating) and homogeneously suspended/cultured in 7% GelMA, fuse together within 2 days of culture in the GelMA (FIG. 18G). The fused cardiac spheroids start to beat together synchronously on day 7 post-cardiac differentiation, and they further merge into each other and beat synchronously and strongly as a whole with further culture in the GelMA to day 10. These data indicate the great potential of the cardiac spheroids differentiated from the eiPSCs 3D-cultured with low RI in 3D for functional 3D cardiac tissue engineering and regenerative medicine applications.

### Example 5: High-Quality 3D Cardiac Differentiation of IMR90-1 iPSCs Cultured in 3D with Low RI

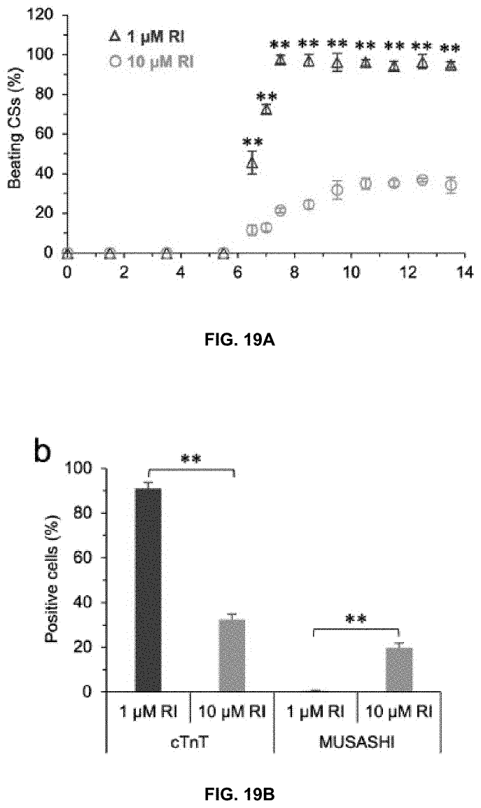

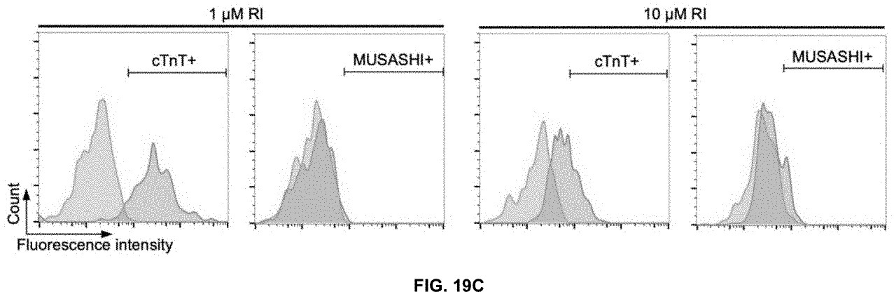

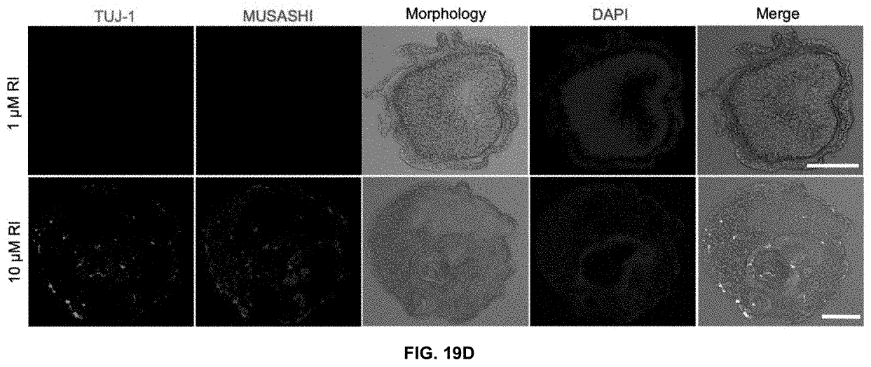

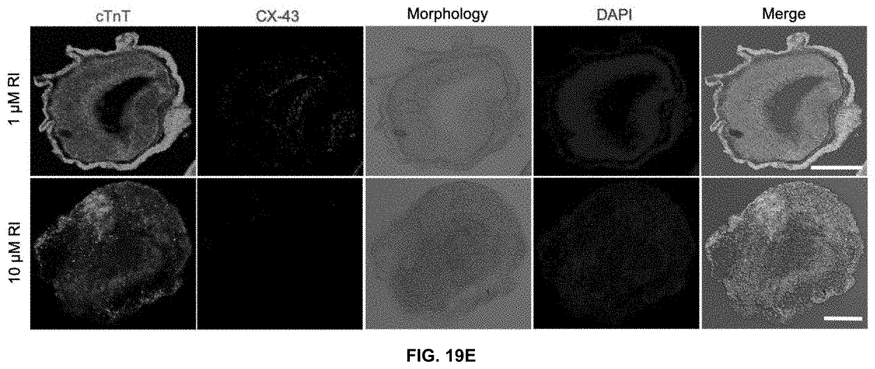

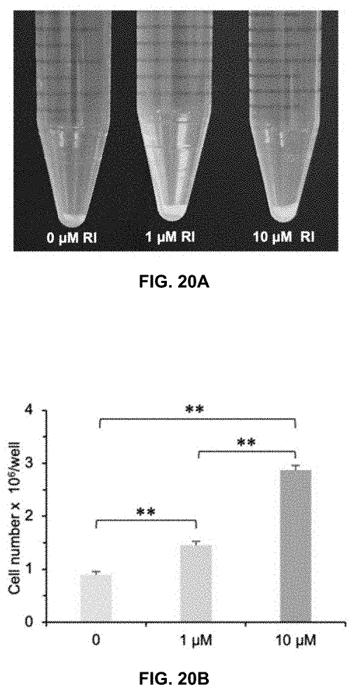

Another commonly utilized iPSC line (IMR90-1) was tested to confirm that the improved cardiac differentiation of iPSCs with low RI for 3D culture before differentiation is not because of the eiPSCs used. The beating of the resultant IMR90-1 cardiac spheroids peaked at ˜98% on day 7.5 with the OBT being synchronized within ˜1 day for the 1 μM RI group (FIG. 19A). In contrast, only 11.5% of the IMR90-1 cardiac spheroids beat on day 7.5 and the beating percentage increases to 34.1% in a week, although it does not seem to change significantly after approximately day 10 for the 10 μM RI group. This was consistent with the flow cytometry data of the cells in the cardiac spheroids collected on day 12.5, showing that 91% and 32% of the cells were positive for the cardiac-specific protein cTnT (that are crucial for beating) in the 1 and 10 μM RI groups, respectively (FIG. 19B-C). Furthermore, cells in the cardiac spheroids that were positive for neural-specific maker MUSASHI were negligible in the 1 μM RI group, while ˜20% cells in the cardiac spheroids from the 10 μM RI group were MUSASHI positive (FIG. 19B-C). The undesired neural differentiation in the 10 μM RI group was further confirmed by immunostaining data showing the existence of MUSASHI- and TUJ-1-positive cells in the cardiac spheroids collected on day 12.5, while the expression of both neural markers was negligible in the cardiac spheroids from the 1 μM RI group (FIG. 19D). Moreover, the expression of the cardiac-specific protein cTnT was high with an evident expression of the gap junction protein CX-43 in the cardiac spheroids of the 1 μM RI group, while the expression of cTnT and CX-43 was low and negligible, respectively, in the cardiac spheroids of the 10 μM RI group (FIG. 19E). Again, these data from the IMR90-1 iPSCs confirm the observation that using a high RI in 3D culture to obtain iPSC spheroids causes heterogeneous lineage-commitment, which compromises the efficiency and quality of their subsequent cardiac differentiation.

### Materials and Methods for Examples 1-5

### Cell Culture

The human eiPSCs (DF19-9-11T.H) and IMR90-1 human iPSCs were cultured in Matrigel-coated 6-well plate in an iPSC maintenance medium made of DMEM/F12 supplemented with bFGF (120 ng/ml), TGF-β (1 ng/ml), γ-aminobutyric acid (100 μg/ml), LiCl 30 (μg/ml), L-glutamine (100 μg/ml), MEM non-essential amino acid (NEAA) solution (0.5%), NaHCO₃ (500 μg/ml), chemically defined lipid concentrate (1%, Invitrogen), sodium selenite (50 ng/ml), bovine serum albumin (20 mg/ml), β-mercaptoethanol (4 μl per 500 ml medium). The cells were passaged twice a week at a ratio between 1:4 and 1:5 with Versene consisting of 0.48 nM ethylenediamineetraacetic acid (EDTA) in 1× (by default) phosphate buffered saline (PBS).

To obtain 3D iPSC spheroids, the iPSC colonies under 2D culture at ˜80% confluence were treated with Versene for 2 min, rinsed with PBS, and further detached from the substrate by gentle pipetting. The detached iPSCs were re-suspended in mTeSR1 with Rock inhibitor (Y27632) at 0, 1, 5, or 10 μM. Afterward, the medium was supplemented with 0.35% (v/v) methylcellulose. The cell suspension (12 ml) containing ˜4×10⁶ cells was pushed through a cell strainer with 100 μm mesh size. Later, the suspension of iPSC clumps was transferred into a petri dish (diameter: 10 cm) for culture in a humidified incubator at 37° C. and 5% CO₂ for 2 days, during which the 2D iPSC clumps grew into 3D iPSC spheroids. Then, the medium was changed to mTeSR1 (supplemented with 0.35% methylcellulose) with no rock inhibitor, to further culture for 2 days before cardiac differentiation. To determine the number of cells per eiPSC spheroid, roughly equal numbers of the spheroids from either the 1 or 10 μM RI group were pooled together and trypsinized to dissociate the cells. The dissociated cells were then resuspended in 1.4 ml of the aforementioned iPSC maintenance medium and the cell number was counted using a hemocytometer. Three independent runs with 282, 260, and 256 spheroids for the 1 μM RI group and 273, 298, and 286 spheroids for the 10 μM RI group were conducted.

### Teratoma Assay

To test the pluripotency of the cells in the 3D iPSC spheroids in vivo with the teratoma assay, the eiPSC spheroids were directly injected subcutaneously (s.c.) into the dorsal rear flank of severe combined immunodeficient mice (NOD.CB17-scid). A total of 2×10⁶ cells in 300 μL of PBS was injected into each mouse (age: 5 weeks, and 5 mice per group). After 5 weeks, the mice were sacrificed, and the resulting teratoma were collected and fixed in 4% paraformaldehyde (PFA) for 3 days. Afterward, the samples were cut into small pieces of ˜0.5 cm³, embedded in paraffin, and sectioned into slices of 5 μm thick. The slices were stained with hematoxylin and eosin (H&E) and imaged with a Zeiss LSM 710 microscope. All animal studies were approved by the Institutional Animal Care and Use Committee (IACUC, #R-MAY-18-24) at the University of Maryland, College Park.

### Cardiac Differentiation in 3D

The eiPSC spheroids obtained after 4 days of culture on day 0 were induced into mesoderm by up-regulation of the Wnt signaling pathway using 8 μM CHIR99021 and 2 μM GSK inhibitor 6-bromoindirubin-3′-oxime (BIO) in the mesoderm induction medium for 1 day. The mesoderm induction medium was a mixture of DMEM/F12 and α-MEM (v/v, 1:1), containing 2% Knockout Serum Replacement (KOSR), 1 mM L-glutamine, 1% MEM non-essential amino acids (NEAA), and 0.1 mM β-mercaptoethanol. After 1 day, the spheroids were induced for cardiac commitment by down-regulating the Wnt signaling pathway using 10 μM KY02111 and 10 μM XAV939 in the cardiac maintenance medium for 6 days with the medium being changed every other day. The cardiac maintenance medium was a mixture of RPMI1640 and α-MEM (v/v, 1:1) containing 5% fetal bovine serum (FBS). Starting from day 8, the cardiac maintenance medium without KY02111 and XAV939 was used, and it was changed every other day for cardiac maturation. Images and videos of the resultant cardiac spheroids were taken using the Zeiss LSM 710 microscope before medium change.

### Cryosectioning and Immunostaining

The iPSC spheroids and the iPSC-derived Cardiac spheroids were fixed in 4% PFA in 1×PBS at 4° C. overnight. The fixed spheroids were incubated sequentially in 10% and 15% sucrose solutions in saline for 4 h, respectively. Afterward, the spheroids were put in a plastic box and embedded in OCT for cryosectioning. Slices of the spheroids of 10 μm thick were obtained by cutting the frozen sample on a Leica cryostat platform and then immediately attached onto the Leica Apex high adhesive glass slides.

For immunostaining, the slides were gently rinsed twice with 1×PBS to remove the OCT and then incubated with 0.1% Tween-20 and 5% normal goat serum in 1×PBS for 1 h at room temperature (RT) to block non-specific bindings. Later, the samples were incubated with primary antibodies at 4° C. overnight. The dilution and product information of the primary antibodies were as follows: OCT-4 (1:500 dilution, Cell Signaling Technologies), NANOG (1:500 dilution, Cell Signaling Technologies), SSEA-4 (1:500 dilution, Cell Signaling Technologies), cTnT (1:500 dilution, Cell Signaling Technologies), CONNEXIN-43 (CX-43, 1:400 dilution, Santa Cruz Biotechnology), DESMIN (1:500 dilution, Santa Cruz Biotechnology), α-ACTININ (1:500 dilution; Santa Cruz Biotechnology), BRACHYURY (1:500; Santa Cruz Biotechnology), NKX2.5 (1:500; Santa Cruz Biotechnology), NESTIN (1:500; R & D Systems), MUSASHI-1 (1:500; R & D Systems), and TUJ-1 (1:500; R & D Systems). Afterward, the samples were rinsed with 1×PBS thrice and then incubated with the associated secondary antibodies (goat anti-rabbit IgG FITC and goat-anti-mouse IgG PE, Invitrogen) in 1×PBS for 1.5 h at RT. Lastly, the samples were rinsed with 1 ml of 1×PBS for 3 min and the nuclei were stained with 1 μg/ml DAPI solution for 5 min at RT. The samples were imaged with the Zeiss LSM 710 microscope.

### Scanning Electron Microscopy

For scanning electron microscopy (SEM), the eiPSC spheroids collected on day −3 were fixed by 4% PFA in 1×PBS at 4° C. overnight. Then, the spheroids were incubated in 15% sucrose solution in saline for 4 h. Afterward, the spheroids were suspended in 100% ethanol and loaded on the SEM sample carrier and dried at RT overnight. The samples were then sputter-coated with gold at 15 mA for 2 min using the Ted Pella Cressington-108 sputter coater. SEM images of the spheroids were obtained with a Hitachi SU-70 FEG scanning electron microscope.

### Flow Cytometry

For flow cytometry, the eiPSC spheroids were collected on day 0 and the cardiac spheroids were collected on days 1.5, 2.5, 12.5, 15. The spheroids were dissociated to single cells by 0.25% trypsin for 5 min at 37° C. and then fixed with 75% ethanol at 4° C. overnight. The cells were permeabilized with 0.05% Triton X-100 for 3 min and then rinsed with 1×PBS twice. The cell numbers were adjusted to 1×10⁶ cells/tube in 700 μL of saline for each marker. These cells were incubated with primary antibodies including OCT-4 (1:500 dilution, Cell Signaling Technologies), NANOG (1:500 dilution, Cell Signaling Technologies), SSEA-4 (1:500 dilution, Cell Signaling Technologies), BRACHYURY (1:500; Santa Cruz Biotechnology), NKX2.5 (1:500; Santa Cruz Biotechnology), cTnT (1:500 dilution, Cell Signaling Technologies), cTnI (1:500 dilution, Cell Signaling Technologies), α-ACTININ (1:500 dilution; Santa Cruz Biotechnology), MUSASHI-1 (1:500; R&D Systems), and TUJ-1 (1:500; R&D Systems) at 4° C. overnight. Subsequently, the samples were rinsed with 1×PBS thrice before incubation with corresponding secondary antibodies: goat anti-mouse IgG FITC and goat anti-rabbit IgG PE, respectively (1:1000; Invitrogen) for 1 h at RT. The samples were then rinsed with 1×PBS thrice before flow cytometry. The negative controls were the cells incubated with respective primary antibodies as aforementioned (no incubation with a secondary antibody).

### RNA Sequencing

For RNA sequencing (RNA-Seq), total RNA was extracted from the eiPSC clumps from 2D culture on day −4 (termed as 2D) and spheroids collected on day −2.5 after 1.5 days in the 3D culture (termed as UO and UT for 1 μM and 10 μM RI conditions, respectively), day 2.5 (termed as CO and CT), and day 13.5 (termed as MO and MT). The DNase I from bovine pancreas was used to remove DNA in samples. The RNA concentration in the samples was measured with Nanodrop and the integrity and quality of RNAs in the samples were confirmed with the Nano RNA Bioanalyzer. Samples with RNA integrity number greater than 9 were used for the preparation of the next-generation sequencing library using the NEB Ultra Directional RNA library preparation kit. Pair-end sequencing was performed by the Illumina HiSeq2500 platform. RNA-seq sequencing data quality was verified by Novogene. Raw reads were mapped to the human reference genome version (hg38) and the gene expression level based on reads per kilobase of exon per million reads mapped for annotated genes was measured and normalized using the Cufflink program. Samples were compared in different combinations and genes with an expression change of more than 1.5 in |log₂ fold change| were defined as differentially expressed genes (DEG). Different classes of genes were subjected to functional and pathway analysis with the Gene Ontology (GO) database.

### Transmission Electron Microscopy

For transmission electron microscopy (TEM) studies, the cardiac spheroids collected on day 12.5 were fixed with 2.5% glutaraldehyde, 2% PFA, and 0.1 M PIPES buffer at pH 7.4 for 2 h at 4° C., rinsed with 1×PBS, and subsequently incubated with 1% osmium tetroxide for 2 h at 4° C. Afterward, the samples were dehydrated by a series of ethanol solutions (75%, 85%, 95%, and 100%) and acetone sequentially. Then, the samples were embedded in the resin EMbed 812 by following the manufacturer's instructions. Slices (70 nm-thick) of the embedded samples were cut with a Leica UC6 ultramicrotome and subsequently stained with 1% (w/v) uranyl acetate for 10 min at RT. The slices were examined and imaged with an FEI Tecnai T12 transmission electron microscope. The lengths of myofibrils and sarcomeres were measured with the NIH ImageJ (v1.52a).

### Calcium Spike Assay

To quantify the calcium spike, the cardiac spheroids collected on day 12.5 either without or with drug treatment were incubated with 2 nM Fluo-4 probes in cardiac maintenance medium for 30 min. Then, the medium was replaced with a fresh cardiac maintenance medium for 20 min. Afterward, the cardiac spheroids were transferred into a 1 cm diameter glass-bottom dish containing 500 μL of cardiac maintenance medium and incubated in the stage incubator of the Zeiss LSM 710 microscope at 37° C. and 5% CO₂ for imaging and video recording. Cardiac drugs Isoproterenol (ISO) and propranolol (PRO) that increase and decrease the heart beating rate, respectively, were used to test the drug response of the cardiac spheroids. To do this, a cardiac maintenance medium containing 10 NM ISO was incubated with the cardiac spheroids for 10 min. After acquiring the calcium spike activity with the ISO treatment, the medium with ISO was removed and the cardiac spheroids were rinsed with fresh cardiac maintenance medium for 3 min. Then, the cardiac spheroids were incubated with a cardiac maintenance medium containing 10 μM PRO for 10 min and the calcium spike activity was recorded. All the video-recording was done at 5 frames per second for 1 min using the Zeiss LSM 710 microscope and analyzed with the Zeiss Zen Blue software. Calcium spike activities were collected from three independent experiments to quantify the beating frequency/rate of the cardiac spheroids. The data of the calcium spikes of the cardiac spheroids were presented as ΔF/F₀, where F represents fluorescence intensity, F₀ is the fluorescence intensity at the resting state of the cardiac spheroids, and ΔF (=F−F₀) is the change of fluorescence intensity.

### Culture of Cardiac Spheroids in Gelatin Methacryloyl Hydrogel

To synthesize gelatin methacryloyl (GelMA), type A porcine skin gelatin (300 bloom) was dissolved at 10% (w/v) into 1×PBS at 50° C. for 20 min. Methacrylic anhydride (MA) was added dropwise into the gelatin solution under vigorous stirring for 1 h (0.6 g of MA per gram of gelatin). The mixture was diluted with 1×PBS to stop the reaction and centrifuged at 2000 g for 2 min. To remove excess acid, the supernatant containing dissolved GelMA was collected and dialyzed (10 kDa molecular weight cutoff) against water. The dialyzed GelMA was then frozen, lyophilized, and stored at −80° C. To make the GelMA solution suspended with cardiac spheroids, the lyophilized GelMA was dissolved at 7% (w/v) in the cardiac maintenance medium at 50° C. for 20 mins. Irgacure 2959 (0.1% (w/v)) was added into the GelMA solution at 50° C. and stirred for 15 min. The resultant GelMA solution was slowly cooled to 37° C. before mixing it with the cardiac spheroids collected on day 5 post-cardiac differentiation (before beating). The GelMA solution suspended with cardiac spheroids was transferred into a well of 12-well plate at 4×10⁶ cells in 0.5 ml of the GelMA solution. The GelMA solution was crosslinked into GelMA hydrogel by exposing it to ultraviolet light at 5 mW cm⁻² for 1 min. Lastly, 1 ml of the cardiac maintenance medium was added into the well and the medium was changed every other day.

### Statistical Analysis

All quantitative data were collected from at least three independent experiments. The data were presented as mean±standard deviation. Student's t-test (two-tails, unpaired, and assuming equal variance) was performed for comparisons between two groups. A p value less than 0.05 was considered to be statistically significant.

What is claimed is:

1. A method of maintaining pluripotency of stem cells in three-dimensional (3D) culture, the method comprising:
culturing pluripotent stem cells in suspension to form an aggregate in a medium comprising 5 μM or less of a Rock inhibitor.

2. The method of claim 1, wherein the medium comprises from about 0.01 μM to about 5 μM of the Rock inhibitor.

3. The method of claim 1, wherein the pluripotent stem cells are induced pluripotent stem cells.

4. The method of claim 1, wherein the medium comprises a viscosity enhancer.

5. The method of claim 4, wherein the medium is mTeSR1 or StemFlex supplemented with the Rock inhibitor and methylcellulose.

6. The method of claim 1, wherein prior to the culturing step, the pluripotent stem cells are cultured in a maintenance media on a substrate.

7. The method of claim 6, wherein the maintenance medium comprises DMEM/F12 supplemented with bFGF, TGF-β, γ-aminobutyric acid, LiCl, L-glutamine, MEM non-essential amino acids, NaHCO₃, chemically defined lipid concentrate, sodium selenite, bovine serum albumin, and β-mercaptoethanol.

8. The method of claim 1, further comprising culturing the aggregate of pluripotent stem cells in suspension in a medium without Rock inhibitor.

9. The method of claim 8, further comprising inducing differentiation of the aggregate of pluripotent stem cells.

10. The method of claim 9, wherein the pluripotent stem cells are differentiated into cardiomyocytes.

11. A method of producing cardiomyocytes or a cardiac organoid, the method comprising:
(a) culturing pluripotent stem cells in suspension to form an aggregate in a medium comprising 5 μM or less of a Rock inhibitor;
(b) culturing the aggregate of pluripotent stem cells in suspension in a medium without Rock inhibitor; and
(c) inducing cardiac differentiation of the aggregate of pluripotent stem cells, thereby producing cardiomyocytes or the cardiac organoid.

12. The method of claim 11, wherein the medium of step (a) comprises from about 0.01 μM to about 5 μM of the Rock inhibitor.

13. The method of claim 11, wherein inducing cardiac differentiation comprises
culturing the aggregate of pluripotent stem cells in a medium comprising a Wnt signaling activator; and
culturing the aggregate of pluripotent stem cells in a medium comprising a Wnt signaling inhibitor.

14. The method of claim 13, wherein the Wnt signaling activator comprises CHIR99021 and 6-bromoindirubin-3′-oxime (BIO).

15. The method of claim 13, wherein the Wnt signaling inhibitor comprises XAV939 and KY02111.

16. The method of claim 11, wherein the outset beating time (OBT) is synchronized within about 24 hours.

17. The cardiomyocytes or cardiac organoid produced by the method of claim 11.

18. A method for screening an agent for improving or diminishing cardiac function, the method comprising:
contacting the cardiomyocytes or cardiac organoid of claim 17 with the agent; and
measuring at least one response of the cardiomyocytes or cardiac organoid.

19. A therapeutic agent comprising the cardiomyocytes or cardiac organoid of claim 17.

20. A method for treating a disorder of a cardiac tissue, the method comprising:
transplanting the cardiomyocytes or cardiac organoid of claim 17 into a heart of a subject in need of treatment.
